(19) United States
(12) Patent Application Publication
(10) Pub. No.: US 2010/0216886 A1
(43) Pub. Date: Aug. 26, 2010

(54) POLYMORPHS OF N2-(1,1'-BIPHENYL-4-YLCARBONYL)-N1-[2-(4-FLUOROPHENYL)-1,1-DIMETHYLETHYL]-L-ALPHA-GLUTAMINE

(75) Inventors: Qiuxia Wang, Somerville, NJ (US); Jennifer Qianying Liang, Morris Plains, NJ (US); Mannching Sherry Ku, Thiells, NY (US); Ronald Stanley Michalak, Congers, NY (US); Wen James Huang, Hillsborough, NJ (US)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/514,114

(22) PCT Filed: Nov. 9, 2007
(86) PCT No.: PCT/US07/84314

§ 371 Date: Jan. 25, 2010

Related U.S. Application Data
(60) Provisional application No. 60/857,779, filed on Nov. 9, 2006.
(60) Provisional application No. 60/857,794, filed on Nov. 9, 2006.
(60) Provisional application No. 60/857,791, filed on Nov. 9, 2006.
(60) Provisional application No. 60/857,781, filed on Nov. 9, 2006.
(60) Provisional application No. 60/857,780, filed on Nov. 9, 2006.
(60) Provisional application No. 60/857,793, filed on Nov. 9, 2006.
(60) Provisional application No. 60/857,792, filed on Nov. 9, 2006.

(51) Int. Cl. A61K 31/197; C07C 229/26; A61P 3/10; A61P 35/00; A61P 37/06; A61P 19/02; A61P 17/02; A61P 27/02; A61P 9/10; A61P 7/06

(52) U.S. Cl. 514/563; 562/450

(57) ABSTRACT

Disclosed are novel polymorphic forms of N²-(1,1′-biphenyl-4-ylcarbonyl)-N¹-[2-(4-fluorophenyl)-1,1-dimethylethyl]-L-α-glutamine, methods of preparing the polymorphic forms, compositions containing the polymorphic forms, and methods of treatment using the polymorphic forms.

### RELATED APPLICATIONS

This application claims priority under 35 USC §119 to U.S. Application Nos. 60/857,794, 60/857,779, 60/857,780, 60/857,781, 60/857,790, 60/857,791, 60/857,792, and 60/857,793, each filed Nov. 9, 2006, the disclosures of which are herein incorporated by reference in their entireties.

Throughout this application, various publications are referenced. The disclosures of these publications in their entireties are hereby incorporated by reference into this application.

This patent disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights whatsoever.

### FIELD OF THE INVENTION

The invention relates to novel polymorphic forms of N²-(1,1′-biphenyl-4-ylcarbonyl)-N¹-[2-(4-fluorophenyl)-1,1-dimethylethyl]-L-α-glutamine, methods of preparing the polymorphic forms, compositions containing the polymorphic forms, and methods of treatment using the polymorphic forms.

### BACKGROUND OF THE INVENTION

N²-(1,1′-biphenyl-4-ylcarbonyl)-N¹-[2-(4-fluorophenyl)-1,1-dimethylethyl]-L-α-glutamine is a modulator of metalloproteinases, as described in U.S. patent application Ser. No. 11/484,005, having Publication No. 2007/0043066, and in International Patent Application No. PCT/US2006/027066, having Publication No. WO 2007/008994, the entire disclosures of which is incorporated herein by reference in their entireties. While the synthesis of N²-(1,1′-biphenyl-4-ylcarbonyl)-N¹-[2-(4-fluorophenyl)-1,1-dimethylethyl]-L-α-glutamine has been described, polymorphic forms of the compound have not been previously described.

Metalloproteinases, including matrix metalloproteinases and aggrecanases, are known to have a role in the breakdown of connective tissue. Matrix metalloproteinases (“MMPs”) constitute a superfamily of proteolytic enzymes that are genetically related and capable of degrading almost all the constituents of extracellular matrix and basement membrane that restrict cell movement. Aggrecanases are members of the ADAMTS (A disintegrin and metalloproteinase with thrombospondin motifs) family of proteins. Aggrecanase-1 and aggrecanase-2 have been designated ADAMTS-4 and ADAMTS-5, respectively (Tang B L, Int J Biochem Cell Biol 2001, 33, 33-44).

The ADAMTS family is involved in cleaving aggrecan, a cartilage component also known as the large aggregating chondroitin sulphate proteoglycan (Abbaszade I et al., J Biol Chem 1999, 274, 23443-23450), procollagen processing (Colige A et al., Proc Natl Acad Sci USA 1997, 94, 2374-2379), angiogenesis (Vazquez F et al., J Biol Chem 1999, 274, 23349-23357), inflammation (Kuno K et al., J Biol Chem 1997, 272, 556-562) and tumor invasion (Masui T. et al., J Biol Chem 1997, 272, 556-562). MMPs have been shown to cleave aggrecan as well.

The loss of aggrecan has been implicated in the degradation of articular cartilage in arthritic diseases. For example, osteoarthritis is a debilitating disease which affects at least 30 million Americans. Degradation of articular cartilage and the resulting chronic pain can severely reduce quality of life. An early and important characteristic of the osteoarthritic process is loss of aggrecan from the extracellular matrix, resulting in deficiencies in the biomechanical characteristics of the cartilage. Likewise, MMPs and aggrecanases are known to play a role in many disorders in which extracellular protein degradation or destruction occurs, such as cancer, asthma, chronic obstructive pulmonary disease (“COPD”), atherosclerosis, age-related macular degeneration, myocardial infarction, corneal ulceration and other ocular surface diseases, hepatitis, aortic aneurysms, tendonitis, central nervous system diseases, abnormal wound healing, angiogenesis, restenosis, cirrhosis, multiple sclerosis, glomerulonephritis, graft versus host disease, diabetes, inflammatory bowel disease, shock, invertebral disc degeneration, stroke, osteopenia, and periodontal diseases.

Therefore, metalloproteinase inhibitors, including inhibitors of MMPs and aggrecanases, are needed.

The present invention is directed to these and other important ends.

### SUMMARY OF THE INVENTION

The invention provides polymorphs of N²-(1,1′-biphenyl-4-ylcarbonyl)-N¹-[2-(4-fluorophenyl)-1,1-dimethylethyl]-L-α-glutamine, including crystalline polymorphs of Form A, Form B, Form C, Form E, Form F, Form G and pseudo Form A, and amorphous polymorph Form D. In some embodiments, the polymorph is a substantially pure polymorph of Form A, Form B, Form C, Form D, Form E, Form F, Form G, or pseudo Form A.

The invention also provides methods for the preparation of the crystalline polymorph of Form A, which comprises dissolving N²-(1,1′-biphenyl-4-ylcarbonyl)-N¹-[2-(4-fluorophenyl)-1,1-dimethylethyl]-L-α-glutamine in an organic solvent to form a solution and evaporating the organic solvent to form the crystalline polymorph of Form A. Crystalline polymorph Form A of N²-(1,1′-biphenyl-4-ylcarbonyl)-N¹-[2-(4-fluorophenyl)-1,1-dimethylethyl]-L-α-glutamine prepared by the methods of the invention is also provided by the invention.

The invention also provides methods for the preparation of the crystalline polymorph of Form B. In some embodiments, the methods comprise converting polymorph Form A to polymorph Form B. In other embodiments, the methods comprise crystallizing polymorph Form B from a mixture of N²-(1,1′-biphenyl-4-ylcarbonyl)-N¹-[2-(4-fluorophenyl)-1,1-dimethylethyl]-L-α-glutamine in a solvent mixture, and isolating the crystalline polymorph Form B. Crystalline polymorph Form B of N²-(1,1′-biphenyl-4-ylcarbonyl)-N¹-[2-(4-fluorophenyl)-1,1-dimethylethyl]-L-α-glutamine prepared by the methods of the invention is also provided by the invention.

The invention also provides methods for the preparation of the crystalline polymorph of Form C. In some embodiments, the methods comprise converting polymorph Form A to polymorph Form C. Crystalline polymorph Form C of N²-(1,1′-biphenyl-4-ylcarbonyl)-N¹-[2-(4-fluorophenyl)-1,1-dimethylethyl]-L-α-glutamine prepared by the methods of the invention is also provided by the invention.

The invention also provides methods for the preparation of the polymorph of Form D. In some embodiments, the methods comprise converting polymorph Form A to polymorph Form D. In other embodiments, the methods comprise equilibrating a slurry of N²-(1,1′-biphenyl-4-ylcarbonyl)-N¹-[2-(4-fluorophenyl)-1,1-dimethylethyl]-L-α-glutamine in a solvent mixture, and isolating the amorphous polymorph Form D. Amorphous polymorph Form D of N²-(1,1′-biphenyl-4-ylcarbonyl)-N¹-[2-(4-fluorophenyl)-1,1-dimethylethyl]-L-α-glutamine prepared by the methods of the invention is also provided by the invention.

The invention also provides methods for the preparation of the crystalline polymorph of Form E. In some embodiments, the methods comprise converting polymorph Form A to polymorph Form E. In other embodiments, the methods comprise equilibrating a slurry of N²-(1,1′-biphenyl-4-ylcarbonyl)-N¹-[2-(4-fluorophenyl)-1,1-dimethylethyl]-L-α-glutamine in a solvent mixture, and isolating the crystalline polymorph Form E. Crystalline polymorph Form E of N²-(1,1′-biphenyl-4-ylcarbonyl)-N¹-[2-(4-fluorophenyl)-1,1-dimethylethyl]-L-α-glutamine prepared by the methods of the invention is also provided by the invention.

The invention also provides methods for the preparation of the crystalline polymorph of Form F, which comprises equilibrating a slurry of N²-(1,1′-biphenyl-4-ylcarbonyl)-N¹-[2-(4-fluorophenyl)-1,1-dimethylethyl]-L-α-glutamine in a solvent, and isolating the crystalline polymorph Form F. Crystalline polymorph Form F of N²-(1,1′-biphenyl-4-ylcarbonyl)-N¹-[2-(4-fluorophenyl)-1,1-dimethylethyl]-L-α-glutamine prepared by the methods of the invention is also provided by the invention.

The invention also provides methods for the preparation of the crystalline polymorph of Form G, which comprises equilibrating a slurry of N²-(1,1′-biphenyl-4-ylcarbonyl)-N¹-[2-(4-fluorophenyl)-1,1-dimethylethyl]-L-α-glutamine in in a solvent mixture, and isolating the crystalline polymorph Form G. Crystalline polymorph Form G of N²-(1,1′-biphenyl-4-ylcarbonyl)-N¹-[2-(4-fluorophenyl)-1,1-dimethylethyl]-L-α-glutamine prepared by the methods of the invention is also provided by the invention.

The invention also provides methods for the preparation of the crystalline polymorph of pseudo Form A, which comprises dissolving N²-(1,1′-biphenyl-4-ylcarbonyl)-N¹-[2-(4-fluorophenyl)-1,1-dimethylethyl]-L-α-glutamine in an organic solvent to form a solution and evaporating the organic solvent to form the crystalline polymorph of pseudo Form A. Crystalline polymorph pseudo Form A of N²-(1,1′-biphenyl-4-ylcarbonyl)-N¹-[2-(4-fluorophenyl)-1,1-dimethylethyl]-L-α-glutamine prepared by the methods of the invention is also provided by the invention.

The invention further provides compositions comprising N²-(1,1′-biphenyl-4-ylcarbonyl)-N¹-[2-(4-fluorophenyl)-1,1-dimethylethyl]-L-α-glutamine of polymorph Form A, Form B, Form C, Form D, Form E, Form F, Form G or pseudo Form A, and a pharmaceutically acceptable carrier. Also provided by the invention are compositions consisting essentially of N²-(1,1′-biphenyl-4-ylcarbonyl)-N¹-[2-(4-fluorophenyl)-1,1-dimethylethyl]-L-α-glutamine of polymorph Form A, Form B, Form C, Form D, Form E, Form F, Form G or pseudo Form A, and a pharmaceutically acceptable carrier. In some embodiments, the pharmaceutically acceptable carrier is suitable for oral administration and the composition comprises an oral dosage form.

In other aspects, the invention provides methods of inhibiting the activity of a metalloproteinase, in an animal in need thereof, which comprises, administering to the animal an effective dose of an inventive composition. In some embodiments, the metalloproteinase is a matrix metalloproteinase or an aggrecanase. In further embodiments, the aggrecanase is aggrecanase-1 or aggrecanase-2. In some embodiments the animal is a mammal, e.g., a mouse, rat, sheep, pig, cow, monkey or human. In some embodiments, the mammal is a human.

In yet other aspects, the invention provides methods for treating a metalloproteinase-related disorder, in an animal in need thereof, which comprises, administering to the animal an effective dose of an inventive composition. In some embodiments, the metalloproteinase is a matrix metalloproteinase or an aggrecanase. In further embodiments, the aggrecanase is aggrecanase-1 or aggrecanase-2. In some embodiments, the metalloproteinase-related disorder is selected from arthritic disorders, osteoarthritis, cancer, rheumatoid arthritis, asthma, chronic obstructive pulmonary disease, atherosclerosis, age-related macular degeneration, myocardial infarction, corneal ulceration and other ocular surface diseases, hepatitis, aortic aneurysms, tendonitis, central nervous system diseases, abnormal wound healing, angiogenesis, restenosis, cirrhosis, multiple sclerosis, glomerulonephritis, graft versus host disease, diabetes, inflammatory bowel disease, shock, invertebral disc degeneration, stroke, osteopenia and periodontal diseases. In some embodiments the animal is a mammal, e.g., a mouse, rat, sheep, pig, cow, monkey or human. In some embodiments, the mammal is a human.

Additional objects and advantages of the invention will be set forth in part in the description which follows, and in part will be apparent from the description, or may be learned by practice of the invention.

### BRIEF DESCRIPTION OF THE FIGURES

FIG. 1 shows a powder XRD (X-ray diffraction) pattern of polymorph Form A.

FIG. 2 shows a powder XRD pattern of polymorph Form B.

FIG. 3 shows a powder XRD pattern of polymorph Form C.

FIG. 4 shows a powder XRD pattern of polymorph Form E.

FIG. 5 shows a powder XRD pattern of polymorph pseudo Form A.

FIG. 6 shows a DSC (differential scanning calorimetry) thermogram of polymorph Form A.

FIG. 7 shows a DSC thermogram of polymorph Form B.

FIG. 8 shows a DSC thermogram of polymorph Form C.

FIG. 9 shows a DSC thermogram of polymorph Form E.

FIG. 10 shows a DSC thermogram of polymorph pseudo Form A.

FIG. 11 shows DSC thermograms of samples of N²-(1,1′-biphenyl-4-ylcarbonyl)-N¹-[2-(4-fluorophenyl)-1,1-dimethylethyl]-L-α-glutamine.

FIG. 12 shows XRD patterns of samples, initially containing polymorph Form A, recovered from various aqueous media.

FIG. 13 shows HPLC (high-performance liquid chromatography) chromatograms of samples of N²-(1,1′-biphenyl-4-ylcarbonyl)-N¹-[2-(4-fluorophenyl)-1,1-dimethylethyl]-L-α-glutamine.

FIG. 14 shows results of a hot stage microscope study of a sample containing polymorph Form B.

FIG. 15 shows XRD patterns of a starting material containing polymorph Form B and the vacuum-dried material.

FIG. 16 shows an XRD pattern showing conversion of polymorph Form B to polymorph Form A at 100° C.

FIG. 17 shows a DVS (Dynamic Vapor Sorption) isotherm plot of a sample containing polymorph Form B.

FIG. 18 shows water adsorption/desorption of a sample containing polymorph Form B at various percentages of relative humidity.

FIG. 19 shows an equilibrium moisture sorption isotherm after 20 days for a sample containing polymorph Form B.

FIG. 20 is a scheme showing the degradation products of N²-(1,1′-biphenyl-4-ylcarbonyl)-N¹-[2-(4-fluorophenyl)-1,1-dimethylethyl]-L-α-glutamine.

FIG. 21 shows a proposed degradation pathway for N²-(1,1′-biphenyl-4-ylcarbonyl)-N¹-[2-(4-fluorophenyl)-1,1-dimethylethyl]-L-α-glutamine.

FIG. 22 shows a solubility and pH profile for N²-(1,1′-biphenyl-4-ylcarbonyl)-N¹-[2-(4-fluorophenyl)-1,1-dimethylethyl]-L-α-glutamine.

FIG. 23 shows a DSC thermogram of polymorph Form C.

FIG. 24 shows a TGA (thermal gravimetric analysis) thermogram of polymorph Form C.

FIG. 25 shows a DSC thermogram showing slow cooling crystallization of N²-(1,1′-biphenyl-4-ylcarbonyl)-N¹-[2-(4-fluorophenyl)-1,1-dimethylethyl]-L-α-glutamine from ethanol.

FIG. 26 shows XRD patterns comparing fast versus slow anti-solvent addition of isopropyl alcohol to a solution of N²-(1,1′-biphenyl-4-ylcarbonyl)-N¹-[2-(4-fluorophenyl)-1,1-dimethylethyl]-L-α-glutamine.

FIG. 27 shows XRD patterns of various polymorphs of N²-(1,1′-biphenyl-4-ylcarbonyl)-N¹-[2-(4-fluorophenyl)-1,1-dimethylethyl]-L-α-glutamine, including Form F (first trace from top) and Form G (second trace from top).

FIG. 28 shows DSC thermograms of various polymorphs of N²-(1,1′-biphenyl-4-ylcarbonyl)-N¹-[2-(4-fluorophenyl)-1,1-dimethylethyl]-L-α-glutamine.

FIG. 29 shows TGA profiles of various polymorphs of N²-(1,1′-biphenyl-4-ylcarbonyl)-N¹-[2-(4-fluorophenyl)-1,1-dimethylethyl]-L-α-glutamine.

FIG. 30 shows TGA and DSC profiles of original N²-(1,1′-biphenyl-4-ylcarbonyl)-N¹-[2-(4-fluorophenyl)-1,1-dimethylethyl]-L-α-glutamine.

FIG. 31 shows TGA and DSC profiles of the product prepared by cooling followed by anti-solvent addition of ethanol and water.

FIG. 32 shows TGA and DSC profiles of the product prepared by anti-solvent addition of isopropyl alcohol in water.

FIG. 33 shows TGA and DSC thermograms of polymorph pseudo Form A.

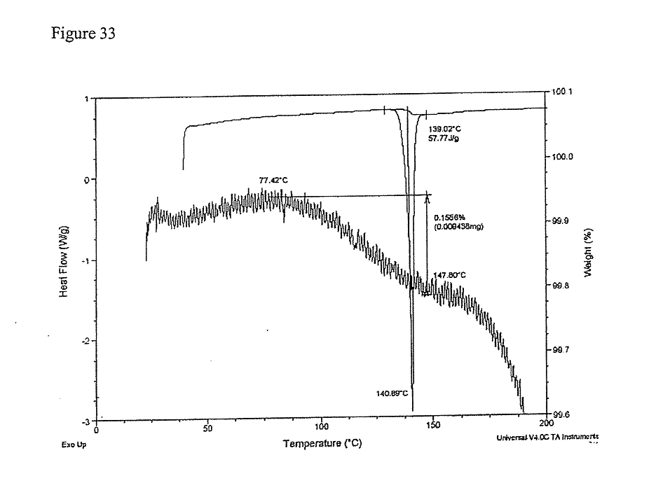

FIG. 34 shows XRD patterns comparing polymorph pseudo Form A with the starting material N²-(1,1′-biphenyl-4-ylcarbonyl)-N¹-[2-(4-fluorophenyl)-1,1-dimethylethyl]-L-α-glutamine.

FIG. 35 shows DSC thermograms comparing polymorph pseudo Form A with the starting material N²-(1,1′-biphenyl-4-ylcarbonyl)-N¹-[2-(4-fluorophenyl)-1,1-dimethylethyl]-L-α-glutamine.

FIG. 36 shows the temperature effect on the rate of transformation from pseudo Form A to a lower melting polymorph form.

FIG. 37 shows the effect of ethanol on the rate of transformation from pseudo Form A to a lower melting polymorph form.

### DETAILED DESCRIPTION OF THE INVENTION

The invention provides for different polymorphs of the compound N²-(1,1′-biphenyl-4-ylcarbonyl)-N¹-[2-(4-fluorophenyl)-1,1-dimethylethyl]-L-α-glutamine, having the formula (I):

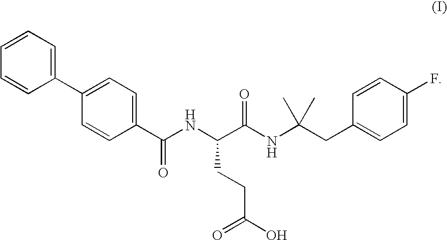

This compound is alternatively named (S)-4-(4-phenylphenylcarbonylamino)-5-oxo-5-(1,1-dimethyl-2-(4-fluorophenyl)-ethylamino)-pentanoic acid.

The invention provides polymorphs of N²-(1,1′-biphenyl-4-ylcarbonyl)-N¹-[2-(4-fluorophenyl)-1,1-dimethylethyl]-L-α-glutamine, including crystalline polymorphs of Form A, Form B, Form C, Form E, Form F, Form G and pseudo Form A, and amorphous polymorph Form D. In some embodiments, the polymorph is a substantially pure polymorph of Form A, Form B, Form C, Form D, Form E, Form F, Form G, or pseudo Form A.

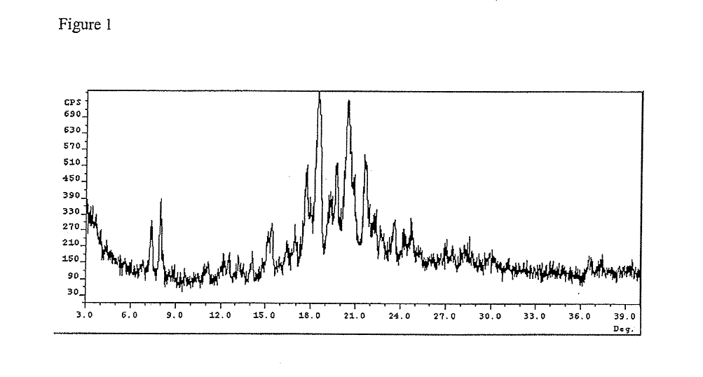

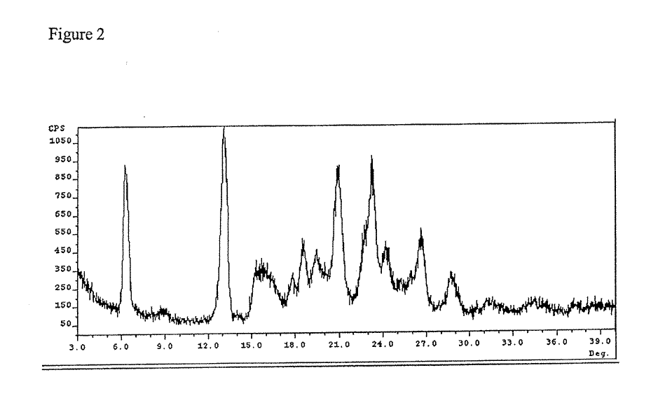

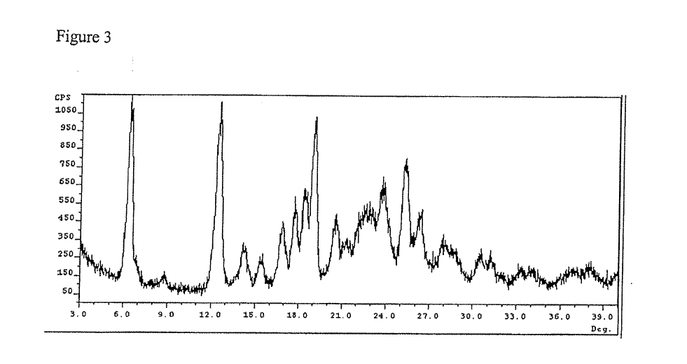

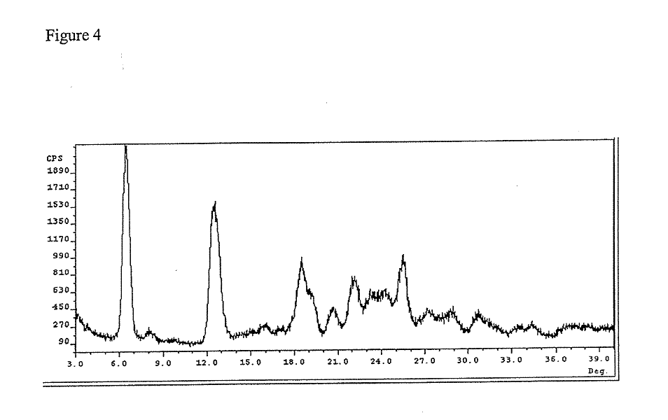

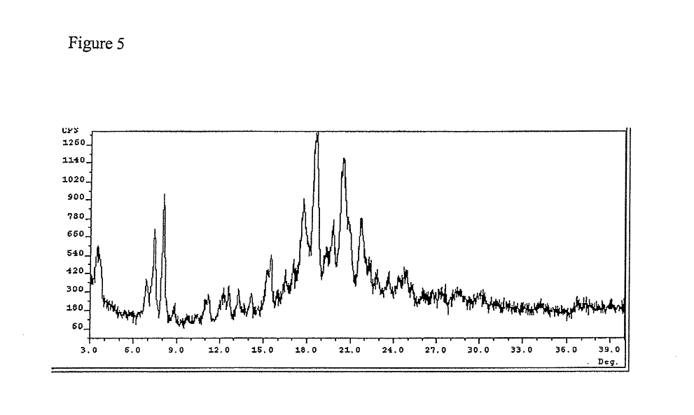

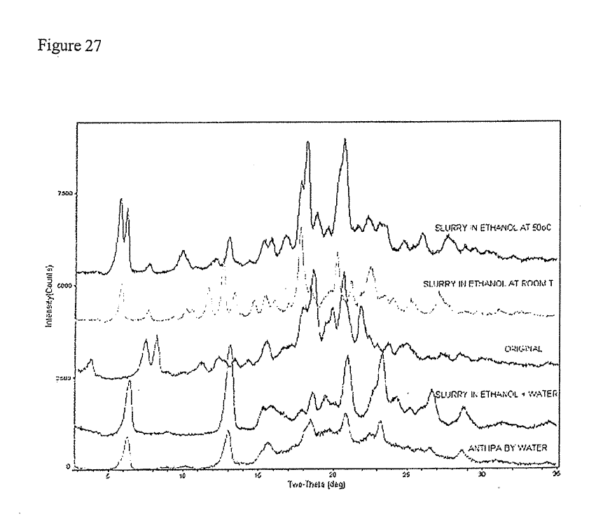

In some embodiments, the polymorph has a powder X-ray diffraction pattern comprising peaks at diffraction angles (degrees 2θ) of about 7.45, 8.01, 15.40, 17.67, 18.49, 19.71 and 20.44 (Form A); of about 6.32, 13.12, 21.01, 23.36, 24.23 and 26.02 (Form B); of about 6.41, 12.54, 14.34, 16.90, 17.80, 19.16, 23.93, 25.40 and 26.52 (Form C); of about 6.44, 12.59, 18.54, 19.09, 22.04 and 25.57 (Form E); of about 5.80, 6.24, 17.84, 18.50, 20.42 and 20.76 (Form F); of about 5.90, 11.50, 13.16, 17.84, 20.20, 21.20, 22.50, and 26.70 (Form G); or of about 7.45, 8.01, 15.17, 17.67, 18.49, 19.71 and 20.44 (pseudo Form A). In some embodiments, polymorph Form A has a powder X-ray diffraction pattern comprising peaks at diffraction angles (degrees 2θ) of about 7.45, 8.01, 15.40, 17.67, 18.49, 19.71, 20.44, and 21.60. In some embodiments, polymorph Form C has a powder X-ray diffraction pattern comprising peaks at diffraction angles (degrees 2θ) of about 6.41, 12.54, 14.34, 16.90, 17.80, 18.50, 19.16, 23.93, 25.40 and 26.52. In some embodiments, polymorph Form F has a powder X-ray diffraction pattern comprising peaks at diffraction angles (degrees 2θ) of about 5.80, 6.24, 10.00, 13.00, 17.50, 18.00, 17.84, 18.50, 20.42 and 20.76. In some embodiments, polymorph Form G has a powder X-ray diffraction pattern comprising peaks at diffraction angles (degrees 2θ) of about 5.90, 11.50, 12.50, 13.16, 17.84, 20.20, 21.20, 22.50, and 26.70. When modifying XRD diffraction angles expressed in degrees 2θ, the term “about” means the stated value ±0.2 degrees. In some embodiments, the polymorph has a powder X-ray diffraction pattern substantially as shown in FIG. 1 (Form A), FIG. 2 (Form B), FIG. 3 (Form C), FIG. 4, (Form E), FIG. 5 (pseudo Form A), or FIG. 27 (Form F, first trace from top; Form G, second trace from top). In some embodiments, the polymorph has a DSC extrapolated melting temperature onset of about 134° C. (Form A), 83° C. (Form B), 83-89° C. (Form C), 80° C. (Form E), 83° C. (Form F), 83° C. (Form G) or 138° C. (pseudo Form A).

The invention also provides methods for the preparation of the crystalline polymorph of Form A, which comprises dissolving N²-(1,1′-biphenyl-4-ylcarbonyl)-N¹-[2-(4-fluorophenyl)-1,1-dimethylethyl]-L-α-glutamine in an organic solvent to form a solution and evaporating the organic solvent to form the crystalline polymorph which is a substantially pure polymorph of Form A. In some embodiments, the organic solvent is selected from: a mixture of ethyl acetate and heptane; toluene; isopropyl acetate; acetonitrile; a mixture of acetone and water; tert-butyl methyl ether; and a mixture of isopropyl acetate and heptane. Crystalline polymorph Form A of N²-(1,1′-biphenyl-4-ylcarbonyl)-N¹-[2-(4-fluorophenyl)-1,1-dimethylethyl]-L-α-glutamine prepared by the methods of the invention is also provided by the invention.

The invention also provides methods for the preparation of the crystalline polymorph of Form B. In some embodiments, the methods comprise equilibrating a slurry of polymorphic Form A in ethanol and water, wherein the polymorph Form A is converted to polymorph Form B, and isolating the crystalline polymorph Form B. In some embodiments, the methods comprise stirring a suspension of polymorph Form A in 2% Tween 80 at about 20-25° C. for about 0.5 hr, wherein the polymorph Form A is converted to polymorph Form B, and isolating the crystalline polymorph Form B. In other embodiments, the methods comprise stirring a suspension of polymorph Form A in water at about 20-25° C. for about 12-24 hr, wherein the polymorph Form A is converted to polymorph Form B, and isolating the crystalline polymorph Form B. In yet other embodiments, the methods comprise crystallizing polymorph Form B from a mixture of N²-(1,1′-biphenyl-4-ylcarbonyl)-N¹-[2-(4-fluorophenyl)-1,1-dimethylethyl]-L-α-glutamine in ethanol and water, ethanol and heptane(s), or isopropanol and water, and isolating the crystalline polymorph Form B. Crystalline polymorph Form B of N²-(1,1′-biphenyl-4-ylcarbonyl)-N¹-[2-(4-fluorophenyl)-1,1-dimethylethyl]-L-α-glutamine prepared by the methods of the invention is also provided by the invention.

The invention also provides methods for the preparation of the crystalline polymorph of Form C. In some embodiments, the methods comprise equilibrating a slurry of polymorphic Form A in ethanol and water, wherein the polymorph Form A is converted to polymorph Form C, and isolating the crystalline polymorph Form C. In other embodiments, the methods comprise equilibrating a slurry of N²-(1,1′-biphenyl-4-ylcarbonyl)-N¹-[2-(4-fluorophenyl)-1,1-dimethylethyl]-L-α-glutamine in ethanol and water, and isolating the crystalline polymorph Form C. Crystalline polymorph Form C of N²-(1,1′-biphenyl-4-ylcarbonyl)-N¹-[2-(4-fluorophenyl)-1,1-dimethylethyl]-L-α-glutamine prepared by the methods of the invention is also provided by the invention.

The invention also provides methods for the preparation of the polymorph of Form D. In some embodiments, the methods comprise equilibrating a slurry of polymorphic Form A in acetone and water, wherein the polymorph Form A is converted to polymorph Form D, and isolating the amorphous polymorph Form D. In other embodiments, the methods comprise equilibrating a slurry of N²-(1,1′-biphenyl-4-ylcarbonyl)-N¹-[2-(4-fluorophenyl)-1,1-dimethylethyl]-L-α-glutamine in acetone and water, and isolating the amorphous polymorph Form D. Amorphous polymorph Form D of N²-(1,1′-biphenyl-4-ylcarbonyl)-N¹-[2-(4-fluorophenyl)-1,1-dimethylethyl]-L-α-glutamine prepared by the methods of the invention is also provided by the invention.

The invention also provides methods for the preparation of the crystalline polymorph of Form E. In some embodiments, the methods comprise equilibrating a slurry of polymorphic Form A in isopropyl alcohol (IPA) and water, wherein the polymorph Form A is converted to polymorph Form E, and isolating the crystalline polymorph Form E. In other embodiments, the methods comprise equilibrating a slurry of N²-(1,1′-biphenyl-4-ylcarbonyl)-N¹-[2-(4-fluorophenyl)-1,1-dimethylethyl]-L-α-glutamine in IPA and water, and isolating the crystalline polymorph Form E. Crystalline polymorph Form E of N²-(1,1′-biphenyl-4-ylcarbonyl)-N¹-[2-(4-fluorophenyl)-1,1-dimethylethyl]-L-α-glutamine prepared by the methods of the invention is also provided by the invention.

The invention also provides methods for the preparation of the crystalline polymorph of Form F, which comprises equilibrating a slurry of N²-(1,1′-biphenyl-4-ylcarbonyl)-N¹-[2-(4-fluorophenyl)-1,1-dimethylethyl]-L-α-glutamine in ethanol at about 50° C., and isolating the crystalline polymorph Form F. Crystalline polymorph Form F of N²-(1,1′-biphenyl-4-ylcarbonyl)-N¹-[2-(4-fluorophenyl)-1,1-dimethylethyl]-L-α-glutamine prepared by the methods of the invention is also provided by the invention.

The invention also provides methods for the preparation of the crystalline polymorph of Form G, which comprises equilibrating a slurry of N²-(1,1′-biphenyl-4-ylcarbonyl)-N¹-[2-(4-fluorophenyl)-1,1-dimethylethyl]-L-α-glutamine in ethanol and water at about 20-25° C., and isolating the crystalline polymorph Form G. Crystalline polymorph Form G of N²-(1,1′-biphenyl-4-ylcarbonyl)-N¹-[2-(4-fluorophenyl)-1,1-dimethylethyl]-L-α-glutamine prepared by the methods of the invention is also provided by the invention.

The invention also provides methods for the preparation of the crystalline polymorph of pseudo Form A, which comprises dissolving N²-(1,1′-biphenyl-4-ylcarbonyl)-N¹-[2-(4-fluorophenyl)-1,1-dimethylethyl]-L-α-glutamine in an organic solvent to form a solution and evaporating the organic solvent to form the crystalline polymorph which is a substantially pure polymorph of pseudo Form A. In some embodiments, the organic solvent is selected from: a mixture of ethyl acetate and heptane; toluene; isopropyl acetate; acetonitrile; a mixture of acetone and water; tert-butyl methyl ether; and a mixture of isopropyl acetate and heptane. Crystalline polymorph pseudo Form A of N²-(1,1′-biphenyl-4-ylcarbonyl)-N¹-[2-(4-fluorophenyl)-1,1-dimethylethyl]-L-α-glutamine prepared by the methods of the invention is also provided by the invention.

Polymorphism is often characterized as the ability of a drug substance to exist as two or more crystalline phases that have different arrangements and/or conformations of the molecules in the crystal lattice. Amorphous solids consist of disordered arrangements of molecules and do not possess a distinguishable crystal lattice. Solvates are crystalline solid adducts containing either stoichiometric or nonstoichiometric amounts of a solvent incorporated within the crystal structure. If the incorporated solvent is water, the solvates are also commonly known as hydrates.

As used herein, “polymorphs” refer to different polymorphic forms of the same compound and includes, but is not limited to, other solid state molecular forms including solvation products and amorphous forms of the same compound. The term “polymorph” refers to any one such form. Different polymorphs of a given compound may differ from each other with respect to one or more physical properties, such as solubility and dissociation, true density, crystal shape, compaction behavior, flow properties, and/or solid state stability. Unstable polymorphs generally convert to the more thermodynamically stable forms at a given temperature after a sufficient period of time. Metastable forms are unstable polymorphs that slowly convert to stable forms. A metastable pharmaceutical solid form can change crystalline structure or solvate/desolvate in response to changes in environmental conditions, processing, or over time. In general, the stable form exhibits the highest melting point and the most chemical stability; however, metastable forms may also have sufficient chemical and physical stability to render them pharmaceutically acceptable. “Chemical stability” refers to stability in chemical properties, such as thermal stability, light stability, and moisture stability.

The different polymorphs of compound (I) include: Polymorph Form A, a higher melting point form and anhydrous; Polymorph Form B, a lower melting point form and a monohydrate; Polymorph Form C, a lower melting point form and a sesquihydrate; a pseudo Form A, and Polymorph Forms D, E, F, and G. It has been surprisingly found that the higher melting point Form A is less stable than lower melting point Form B.

Except as otherwise indicated, the term “about” modifying a value means the nominal value ±3%. Furthermore, the recitation of “about” preceding a series of values is intended to modify each value in the series, e.g., “about 7.45, 8.01, 15.40, 17.67, 18.49, 19.71 and 20.44” is equivalent to “about 7.45, about 8.01, about 15.40, about 17.67, about 18.49, about 19.71 and about 20.44”. Similarly, the recitation of “about” preceding a range of values is intended to modify both endpoints in the range, e.g., “about 83-89° C.” is equivalent to “about 83° C. to about 89° C.”.

The polymorph forms of the invention are preferentially substantially pure, meaning each form contains less than 15%, preferably less than 10%, preferably less than 5%, preferably less than 1% by weight of impurities, including other polymorphic forms of compound (I). Some embodiments provided by the invention are compositions wherein at least 50% by weight of the total of N²-(1,1′-biphenyl-4-ylcarbonyl)-N¹-[2-(4-fluorophenyl)-1,1-dimethylethyl]-L-α-glutamine in the composition is present as the crystalline polymorph. In further embodiments, at least 70%, at least 80%, or at least 90% by weight of the total of N²-(1,1′-biphenyl-4-ylcarbonyl)-N¹-[2-(4-fluorophenyl)-1,1-dimethylethyl]-L-α-glutamine in the composition is present as the crystalline polymorph. Also provided by the invention are compositions consisting essentially of N²-(1,1′-biphenyl-4-ylcarbonyl)-N¹-[2-(4-fluorophenyl)-1,1-dimethylethyl]-L-α-glutamine wherein at least 97-99% by weight of the N²-(1,1′-biphenyl-4-ylcarbonyl)-N¹-[2-(4-fluorophenyl)-1,1-dimethylethyl]-L-α-glutamine is present in the composition as the crystalline polymorph. The polymorph forms of the invention can also be present in mixtures.

The invention further provides compositions comprising N²-(1,1′-biphenyl-4-ylcarbonyl)-N¹-[2-(4-fluorophenyl)-1,1-dimethylethyl]-L-α-glutamine of polymorph Form A, Form B, Form C, Form D, Form E, Form F, Form G or pseudo Form A, and a pharmaceutically acceptable carrier. In some embodiments, at least 50% by weight of the total of N²-(1,1′-biphenyl-4-ylcarbonyl)-N¹-[2-(4-fluorophenyl)-1,1-dimethylethyl]-L-α-glutamine in the composition is present as the polymorph. In further embodiments, at least 70%, at least 80%, or at least 90% by weight of the total of N²-(1,1′-biphenyl-4-ylcarbonyl)-N¹-[2-(4-fluorophenyl)-1,1-dimethylethyl]-L-α-glutamine in the composition is present as the polymorph. Also provided by the invention are compositions consisting essentially of N²-(1,1′-biphenyl-4-ylcarbonyl)-N¹-[2-(4-fluorophenyl)-1,1-dimethylethyl]-L-α-glutamine of polymorph Form A, Form B, Form C, Form D, Form E, Form F, Form G or pseudo Form A, and a pharmaceutically acceptable carrier. In some embodiments, the pharmaceutically acceptable carrier is suitable for oral administration and the composition comprises an oral dosage form.

The polymorphs of N²-(1,1′-biphenyl-4-ylcarbonyl)-N¹-[2-(4-fluorophenyl)-1,1-dimethylethyl]-L-α-glutamine differ in their crystal structure as determined by powder X-ray crystallography. FIGS. 1-5 and 27 show powder X-ray diffraction patterns for the various polymorphic forms, and Table 1 lists the diffraction patterns for the various polymorphic forms, expressed in terms of the degrees 2-theta (2θ).

TABLE 1

| Form A | Form B | Form C | Form E | Form F | Form G | Pseudo A |
|---|---|---|---|---|---|---|
| 7.45 | 6.32 | 6.41 | 6.44 | 5.80 | 5.90 | 7.45 |
| 8.01 | 13.12 | 12.54 | 12.59 | 6.24 | 11.50 | 8.01 |
| 15.40 | 21.01 | 14.34 | 18.54 | 17.84 | 13.16 | 15.17 |
| 17.67 | 23.36 | 16.90 | 19.09 | 18.50 | 17.84 | 17.67 |
| 18.49 | 24.23 | 17.80 | 22.04 | 20.42 | 20.20 | 18.49 |
| 19.71 | 26.02 | 19.16 | 25.57 | 20.76 | 21.20 | 19.71 |
| 20.44 | | 23.93 | | | 22.50 | 20.44 |
| | | 25.40 | | | 26.70 | |
| | | 26.52 | | | | |

X-ray powder diffraction patterns of solid phases were recorded with a Scintag Advanced Diffraction System X2 using Cu KR radiation, a tube voltage of 45 kV, and a tube current of 40 mA. The intensities were measured from 3° to 45° at a continuous scan rate of 4.5°/min.

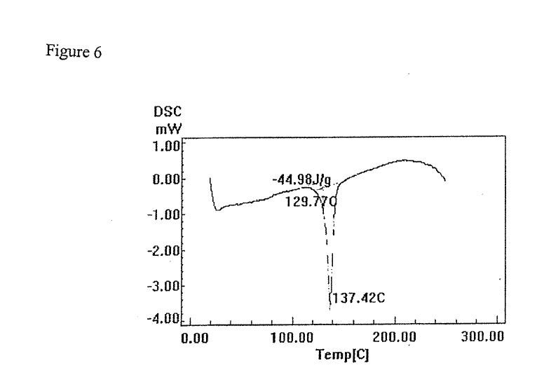

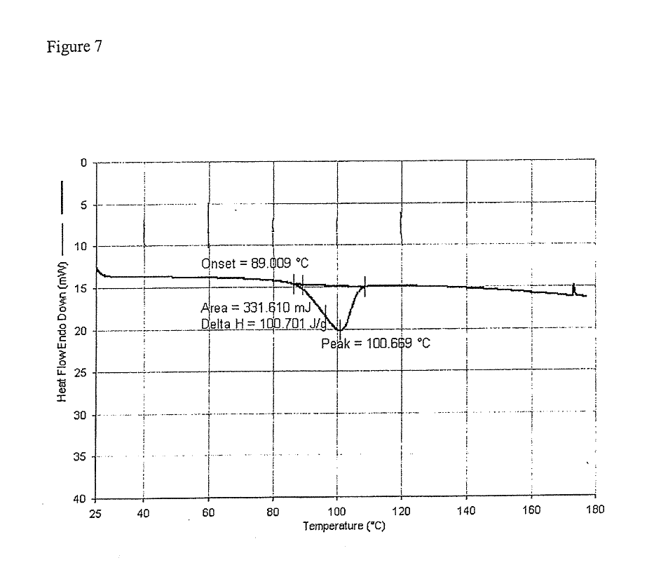

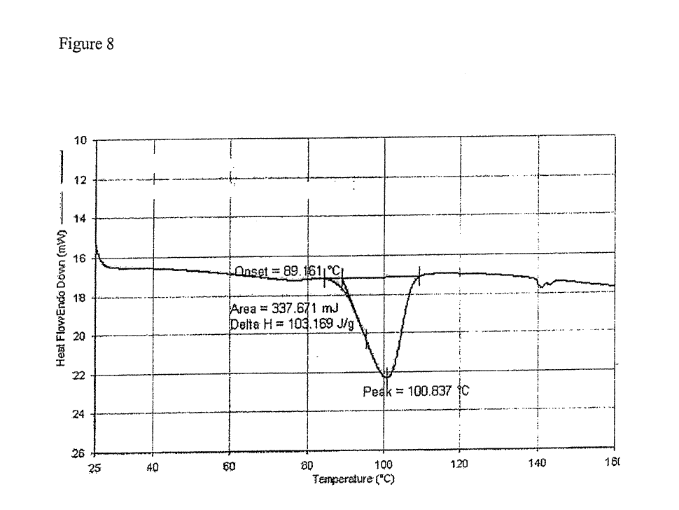

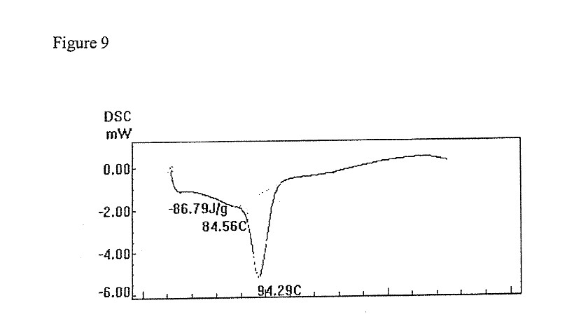

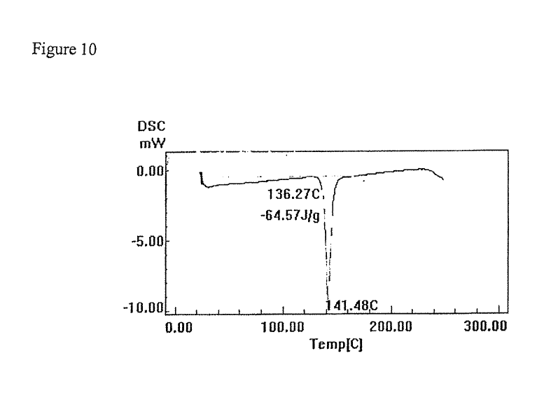

The polymorphs of N²-(1,1′-biphenyl-4-ylcarbonyl)-N¹-[2-(4-fluorophenyl)-1,1-dimethylethyl]-L-α-glutamine also differ in their DSC (differential scanning calorimetry) onset of melting temperatures, as determined by a Shimadzu D50 instrument, at a scan rate of 10° C. per minute. Depending on the rate of heating, i.e. scan rate, at which the DSC analysis is conducted, the calibration standard used, instrument calibration, the relative humidity and upon the relative purity, the endotherms of the polymorphs may vary by about 0.01-10° C., or about 0-5° C., above or below the determined endotherms. The observed endotherm may also differ from instrument to instrument for any given sample. In some embodiments, the crystalline polymorph of Form B has a DSC extrapolated melting temperature onset of about 80-89° C., or about 134-138° C. FIGS. 6-10 show DSC thermograms for the various polymorphic forms.

Polymorphs of the invention may be obtained by crystallization from a solution or slurry of compound (I), with each polymorph resulting by crystallization from a different crystallization solvent. As used herein, a “crystallization solvent” refers to a solvent or combination of solvents used to crystallize a polymorph of compound (I) to preferentially form the substantially pure polymorph form. In some embodiments, the crystallization solvent can be seeded with one or more crystals of a particular polymorph in order to promote formation of that particular crystal in the crystallization solvent.

Polymorphs of the invention may also be obtained by recrystallization from a solution or slurry containing a different form of the polymorph. For example, polymorph Form B can be obtained by recrystallizing polymorph Form A in an appropriate solvent.

For purposes of administration, a polymorph of the invention may be formulated as a pharmaceutical composition. Pharmaceutical compositions of the invention comprise a polymorph and a pharmaceutically acceptable carrier, wherein the polymorph is present in the composition in an amount that is effective to treat the condition of interest. The concentration of the compounds described herein in a therapeutic composition will vary depending upon a number of factors, including the dosage of the drug to be administered and the route of administration. Appropriate concentrations and dosages can be readily determined by one skilled in the art.

Pharmaceutically acceptable carriers are familiar to those skilled in the art. The compositions can be formulated as liquid solutions, and include carriers such as saline and sterile water. The compositions can also be formulated as pills, capsules, granules, or tablets which contain the polymorph along with diluents, dispersing and surface active agents, binders, and lubricants. One skilled in the art may formulate the compositions in an appropriate manner, and in accordance with accepted practices, such as those described in Remington: The Science and Practice of Pharmacy, 20th edition, Alfonso R. Gennaro (ed.), Lippincott Williams & Wilkins, Baltimore, Md. (2000).

The invention also provides methods of inhibiting the activity of a metalloproteinase. The metalloproteinase can be, for example, a matrix metalloproteinase or an aggrecanase, such as aggrecanase-1 or aggrecanase-2.

The invention further provides methods of treating metalloproteinase-related disorders, such as arthritic disorders, osteoarthritis, cancer, rheumatoid arthritis, asthma, chronic obstructive pulmonary disease, atherosclerosis, age-related macular degeneration, myocardial infarction, corneal ulceration and other ocular surface diseases, hepatitis, aortic aneurysms, tendonitis, central nervous system diseases, abnormal wound healing, angiogenesis, restenosis, cirrhosis, multiple sclerosis, glomerulonephritis, graft versus host disease, diabetes, inflammatory bowel disease, shock, invertebral disc degeneration, stroke, osteopenia, and periodontal diseases. These methods include the step of administering, to an animal in need thereof, an effective dose of a pharmaceutical composition comprising a polymorph of compound (I). In some embodiments the animal is a mammal, e.g., a mouse, rat, sheep, pig, cow, monkey or human. In some embodiments, the mammal is a human.

The methods of the invention include systemic administration of a polymorph as disclosed herein, preferably in the form of a pharmaceutical composition. As used herein, systemic administration includes both oral and parenteral methods of administration. For oral administration, suitable compositions include powders, granules, pills, tablets and capsules as well as liquids, syrups, suspensions and emulsions. These compositions may also include flavorants, preservatives, suspending, thickening and emulsifying agents, and other pharmaceutically acceptable additives. For parental administration, the compounds of the present invention can be prepared in aqueous injection solutions that may contain buffers, antioxidants, bacteriostats, and other additives commonly employed in such solutions.

Additionally, regarding pharmaceutically acceptable carriers and the manufacture of compositions containing N²-(1,1′-biphenyl-4-ylcarbonyl)-N¹-[2-(4-fluorophenyl)-1,1-dimethylethyl]-L-α-glutamine and one or more such carriers, methods of administration, determination of effective doses and the like, reference is made to U.S. patent application Ser. No. 11/484,005, having Publication No. 2007/0043066, and International Patent Application No. PCT/US2006/027066, having Publication No. WO 2007/008994.

The polymorphs of the invention may be synthesized in according with the following non-limiting examples, which are illustrative.

### EXAMPLES

### Example 1

### Synthesis of N²-(1,1′-biphenyl-4-ylcarbonyl)-N¹-[2-(4-fluorophenyl)-1,1-dimethylethyl]-L-α-glutamine

The starting material compound (I) was synthesized in accordance with Example 8OO of U.S. patent application Ser. No. 11/484,005, the disclosure of which is herein incorporated by reference in its entirety, and the following description.

Fmoc-L-Glu-(OtBu)—OH hydrate was combined with toluene. The mixture was stirred and concentrated. After cooling, 2-(4-Fluorophenyl)-1,1-dimethylethylamine hydrochloride, isobutyl chloroformate, and 4-methylmorpholine were added and stirred. The mixture was heated, combined with water and the organic phase was separated. The organic phase was mixed with diethylamine, stirred and concentrated. Heptane(s), hydrochloric acid and water were added and the aqueous phase separated. The aqueous phase was extracted with a mixture of toluene and heptane(s) and further extractions with a mixture of toluene and heptane(s) may be repeated as needed.

The aqueous phase was combined with tert-butyl methyl ether and aqueous potassium carbonate, stirred and the organic phase separated. Optionally, the aqueous phase was back extracted with tert-butyl methyl ether. Organic phases were combined and washed with brine to give a solution of 4-Amino-4-[2-(4-fluorophenyl)-1,1-dimethylethylcarbamoyl]butyric acid tert-butyl ester in tert-butyl methyl ether. Or, the organic phase was dried over anhydrous magnesium sulfate and filtered.

4-Amino-4-[2-(4-fluorophenyl)-1,1-dimethylethylcarbamoyl]butyric acid tert-butyl ester, tert-butyl methyl ether, and triethylamine were combined and stirred. The solution was mixed with a solution of biphenyl-4-carbonyl chloride in THF and stirred. The solution was combined with hydrochloric acid and water, and the organic phase separated. The organic phase was washed with aqueous sodium bicarbonate and water and the organic phase separated. The organic phase was combined with isopropanol, concentrated, cooled, water added, combined with isopropanol, and cooled. The solid product was filtered, washed and dried.

The solid product was mixed with toluene, stirred and cooled. Trifluoroacetic acid was added and the mixture was heated. The mixture was cooled, concentrated, mixed with aqueous potassium acetate, tetrahydrofuran or ethyl acetate and the organic phase separated. The organic phase was combined with aqueous potassium acetate and water and the organic phase separated. THF or ethyl acetate was added as needed.

The organic phase was clarified as needed, then combined with heptane(s) to precipitate N²-(1,1′-biphenyl-4-ylcarbonyl)-N¹-[2-(4-fluorophenyl)-1,1-dimethylethyl]-L-α-glutamine. Alternatively, the organic phase was concentrated, diluted with toluene, filtered, washed and dried to give N²-(1,1′-biphenyl-4-ylcarbonyl)-N¹-[2-(4-fluorophenyl)-1,1-dimethylethyl]-L-α-glutamine.

### Example 2

### Preparation of Polymorph Form A

Anhydrous polymorph Form A was formed by dissolving the starting material compound (I) in an organic solvent to form a solution and evaporating the organic solvent to form the crystalline polymorph. The organic solvent can be a mixture of ethyl acetate (EtAc) and heptane; toluene; isopropyl acetate (IPAc); acetonitrile; a mixture of acetone and water; tert-butyl methyl ether; or a mixture of isopropyl acetate and heptane.

### Example 3

### Preparation of Polymorph Form B

Polymorph Form B can be formed by crystallization from a mixture in ethanol and water, in ethanol and heptane(s), or isopropanol and water.

In one example, N²-(1,1′-biphenyl-4-ylcarbonyl)-N¹-[2-(4-fluorophenyl)-1,1-dimethylethyl]-L-α-glutamine was combined with ethanol, stirred and heated (if needed). The mixture was clarified, mixed with heptane(s) (seeded as needed), additional water added as needed, and stirred. Solid products were washed and dried to give Polymorph Form B. Alternatively, the starting material was combined with water and isopropanol, stirred, filtered, washed and dried to give Polymorph Form B.

### Example 4

### Polymorphic Conversion Study of Polymorph Form A to Polymorph Form B in Various Aqueous Media

4.1 Sample Preparation

(1) Sample A, starting from a Sample E containing polymorph pseudo Form A, stirred in 100 mg/mL in 2% Tween80, solidified to a paste in about 30 minutes, was washed and centrifuged (3 times), then vacuum dried.
(2) Sample B, starting from a Sample E containing polymorph pseudo Form A, stirred in 100 mg/mL in water, solidified to a paste after overnight stirring, and was then vacuum dried.
(3) Sample C, starting from a Sample D containing polymorph Form A, stirred in 100 mg/mL in water overnight, did not form a paste, and was vacuum dried.

4.2 Characterization of the Polymorphs Recovered in Aqueous Media by DSC, XRD and HPLC

Polymorphic conversion of Form A to Form B was not observed with Sample D in 2% Tween 80 or water. Although containing the same polymorph Form A as determined by XRD, Sample D is more stable in aqueous conditions compared to Sample E (Table 2).

TABLE 2
Summary of DSC Data from Different Samples of Compound (I)

| | Sample | | | | |
|---|---|---|---|---|---|
| | A | B | C | D | E |
| Sample/Batch | From Sample | From Sample | From Sample | 139 g | 627 g |
| Size | E in 2% | E in Water, | D in Water, | | |
| | Tween 80, 0.5 hr | overnight | overnight | | |
| Tm (Onset, | 71.5° C. | 81.5° C. | 130.6° C. | 129.8° C. | 135.6° C. |
| DSC) | 135.2° C. | 136.6° C. | | | |
| Polymorph | B | B | A | A | Pseudo A |
| Form | | | | | |

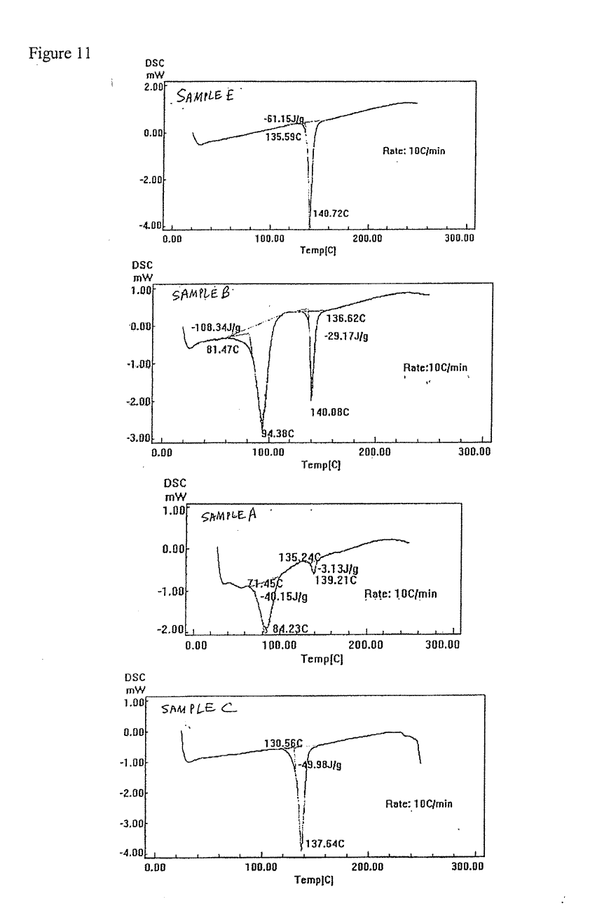

Polymorph pseudo Form A was obtained from a scaled up preparation of polymorph Form A (Sample E). Polymorphic conversions of unstable pseudo Form A to Form B were observed with Sample E. Polymorphic conversions occurred in water (Sample B) and in 2% Tween 80 (Sample A) suspensions as evidenced by changes in XRD patterns and DSC endotherms (FIG. 11).

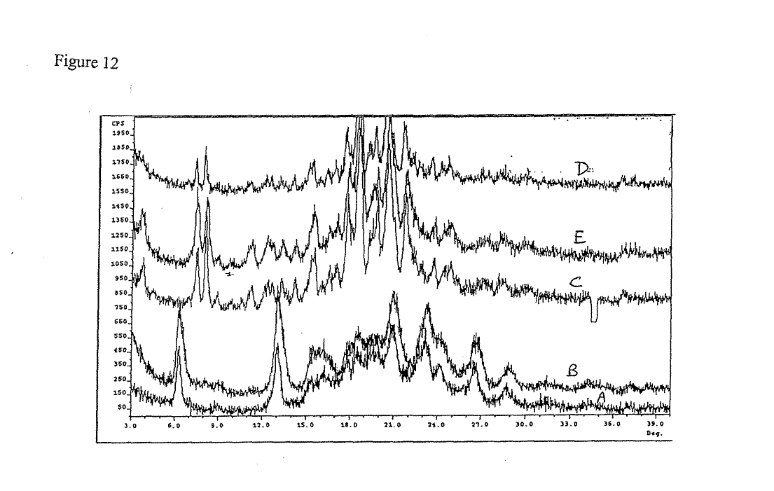

DSC results also confirmed that stirring a suspension of Sample E at ambient conditions for about 0.5 hour in 2% Tween 80 (Sample A), or overnight in a water suspension (Sample B), resulted in the transformation of pseudo Form A to Form B (Table 2, and FIG. 12). The polymorphic conversion appeared to be slower in water than in the 2% Tween suspension, probably due to the lower solubility in water.

Samples D and E showed different stabilities in water. DSC (FIG. 3) and X-Ray diffraction (FIG. 12) results of Sample C confirmed that Sample D did not undergo polymorphic conversion after ˜18 hr in water at ambient conditions.

The XRD pattern of Sample C is similar to the pattern of Sample D, indicating that polymorphic conversion of Form A to Form B did not occur for Sample D in water overnight at ambient conditions. Sample D did not undergo polymorphic conversion overnight in a 2% Tween 80/water suspension. DSC data also supported this observation (FIG. 11).

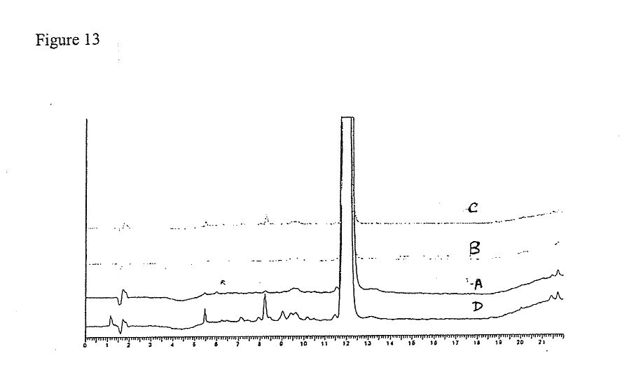

HPLC (high pressure liquid chromatography) chromatograms (FIG. 13) confirmed that the converted materials (Samples A and B) do not contain degradation products of compound (I). The chromatograms (FIG. 13) showed that Sample D has higher impurities (1.97%) compared to Sample C, which indicates that some of the impurities were water soluble and not recovered.

### Example 5

### Polymorph Screening Using Tox Formulation Stability as the Primary Endpoint

5.1 Sample Preparation

Sample E was initially produced as anhydrous Form A by evaporative crystallization from ethyl acetate (EtAc)/heptane without seeding. In order to generate more polymorphic forms, Sample E was slurried in nine different solvents, producing a total of nine 1-g scale batches and one 10-g scale batch. The detailed solvent crystallization information is summarized in Table 3.

Suspensions of 10 to 20 mg/mL of compound (I) in a Tox (toxicological) formulation (2% Tween 80 in water) using the polymorphs described above were prepared and equilibrated at ambient conditions. The stability of the suspensions was evaluated visually and the solubility of the suspensions was determined by HPLC.

5.2 Analytical HPLC Method

### Column: Luna C₁₈ (2) 5 μm, 150×4.6 mm

Mobile Phase: A=900 mL/100 mL/0.1 mL Water/Acetonitrile/TFA

B=100 mL/900 mL/0.1 mL Water/Acetonitrile/TFA

Flow Rate: 1 mL/min

### UV Detection: 270 nm

### Injection Volume: 10 μL

### Temperature: 30° C.

LOD=0.03 μg/mL
LOQ=0.10 μg/mL (RSD=0.27%)

TABLE 4
HPLC parameters

| Time (minute) | % A | % B |
|---|---|---|
| 7.0 | 75 | 25 |
| 2.0 | 45 | 55 |
| 13.0 | 40 | 60 |
| 2.0 | 0.0 | 100 |
| 5.0 | 0.0 | 100 |
| 0.1 | 75 | 25 |

5.3 Results and Discussion

Only the polymorphs obtained from isopropyl alcohol (IPA)/water and acetone/water (Table 3) were stable in the targeted Tox formulation of 2% Tween 80/water. In order to simplify the crystallization process, two more 1 g-scale batches were made (Table 5). The ethanol/water final crystallization step was optimized and the particles processed from 20% ethanol/water were selected for further polymorph screening.

Additional polymorph screening was conducted by using Tox formulation stability as the primary screening method. The crystal particles from Sample 2 (Table 5) crystallized from 30 parts 20% ethanol/water were easier to wet and easier to handle, compared to Sample 7 (Table 3) crystallized from IPA/water, which was sticky and static and hard to handle.

TABLE 3
Summary of Polymorph Selection Studies

| | | Physical | | Melting | | | |
|---|---|---|---|---|---|---|---|
| | Recrystallization | Observation, | H_{f} | Point | Solubility | Suspension | Observation, |
| Sample | Solvent | Solid | (J/g) | (° C.) | (μg/mL) | (mg/mL) | Suspension |
| 1 | EtAc/heptane | White | NA | NA | NA | 16.1 | After 3.5 hr, the |
| | | powder | | | | | suspension turn into |
| | | | | | | | a paste |
| 2 | EtAc/heptane | White | NA | NA | NA | 14.4 | After 4.5 hr, the |
| | | powder | | | | | suspension turn into |
| | | | | | | | a paste |
| 3 | EtAc/heptane | Sticky white | NA | NA | NA | 17.2 | After 1 hr, the |
| | | Powder/solid | | | | | suspension turn into |
| | | | | | | | a curded spoiled |
| | | | | | | | milk like material, |
| | | | | | | | become a paste after |
| | | | | | | | overnight at RT |
| 4 | Acetone/water | Very sticky | −104.0 | 108.7 | 221.3 | 19.3 | Good suspension |
| | | white | | | | | after overnight at RT |
| | | Powder/solid | | | | | |
| 5 | Acetone/heptane | Sticky white | NA | NA | NA | 11.1 | After 0.5 hr, the |
| | | Powder/solid | | | | | suspension turn into |
| | | | | | | | a paste |
| 6 | IPA/heptane | Sticky white | NA | NA | NA | 19.2 | After 0.5 hr, the |
| | | Powder/solid | | | | | suspension turn into |
| | | | | | | | gel, become solid |
| | | | | | | | after overnight at RT |
| 7 | IPA/water | Sticky and | −86.8 | 84.6 | 216.4 | 14.1 | Good suspension |
| | | static white | | | | | after overnight at RT |
| | | Powder/solid | | | | | |

TABLE 5
Samples Using 20% Ethanol/Water As the Final Crystallization Solvent

| | | | | Melting | | | |
|---|---|---|---|---|---|---|---|
| | Recrystallization | Observation | H_{f} | Point | Solubility | Suspension | Observation |
| Sample | Solvent | Solid | (J/g) | (° C.) | (μg/mL) | (mg/mL) | Suspension |
| 1 | Slurry in 20 | White | −118.2 | 76.7 | 201.9 | 17.9 | Good suspension, |
| | parts | pharmaceutical | | | | | after 4.5 hr, and |
| | 5% ethanol/water | powder. Particles | | | | | through |
| | | are hard to wet, | | | | | overnight, tiny |
| | | and hard to | | | | | precipitation was |
| | | suspend. | | | | | observed, and |
| | | | | | | | sticky on the |
| | | | | | | | surface of the |
| | | | | | | | glass. |
| 2 | Slurry in 30 | White | −115.7 | 80.9 | 245.0 | 17.8 | Good suspension, |
| | parts | pharmaceutical | | | | | overnight. |
| | 5% ethanol/water | powder. Particles | | | | | |
| | | are hard to wet, | | | | | |
| | | and hard to | | | | | |
| | | suspend. | | | | | |

Table 6 summarizes various solid-state properties of samples used in toxicological formulation assessments.

TABLE 6

| | Sample | | | | |
|---|---|---|---|---|---|
| | D | G | H | E | F |
| Batch Size (g) | 56 | 127 | 61.9 | 605 | 620 |
| Solubility (mg/mL) | | | | | |
| Water (pH) | 0.037 (5.5) | N/A | N/A | N/A | 0.099 (7.6) |
| 2% Tw80 (pH) | 0.468 (5.4) | 0.127 (5.9) | N/A | 0.330 (5.6) | 0.217 (5.9) |
| TGA Weight Loss | 0.7% | 0.4% | 0.98% | 0.57% | 2.0% |
| (25 to 138 or 150° C.) | | | | | |
| DSC Tₒₙₛₑₜ | 133.5° C. | 134.9° C. | 139.6° C. | 137.3° C. | 83.0° C. |
| Particle Size (μm) | | | | | |
| Malvern | 9.6 (50%) | 6.7 (50%) | 10.7 (50%) | 7.9 (50%) | 6.0 (50%) |
| | 25.7 (90%) | 18.4 (90%) | 29.0 (90%) | 23.1 (90%) | 14.7 (90%) |
| Microscope | NA | N/A | N/A | Mostly Rods | 5.2 (50%) 10.7 (90%) |
| | | | | | Needles |
| Water Content (KF) | 0.17% | 0.41% | 0.27% | 0.06% | 3.54% |
| Crystallinity (XRD) | Pattern A | Pattern A | Pattern A | Pattern A | Pattern B |
| | Form A | Form A | Form A | Pseudo Form A | Form B |
| Total HPLC impurity | 1.97% | 1.56% | 0.04% | 0.28% | 0.25% |
| | (214 nm) | (214 nm) | (214 nm) | (210 nm) | (210 nm) |

The material with XRD pattern B from the ethanol/water system was characterized as polymorph Form B. Sample E, containing polymorph pseudo Form A, was reworked to yield Sample F, containing stable polymorph Form B. The first crop of the reworked Sample E contained more than one mole equivalent of water and was characterized as a sesquihydrate (polymorph Form C), which was subsequently dried to the stoichiometric moisture content of 3.54% for the monohydrate Form B.

Table 7 summarizes various properties of the polymorphs of the invention.

TABLE 7

| XRD Pattern | Form | Solvent | | DSC onset (° C.) | Sample |
|---|---|---|---|---|---|
| A | A | EtAc/Heptane | Anhydrate | ~134 | D, G |
| A | Pseudo Form A | EtAc/Heptane | Anhydrate | ~138 | E, H |
| B | B | EtOH/Water | Monohydrate | 83 | 2 (Table 5), F |
| C | C | EtOH/Water | Sesquihydrate | 83-89 | I |
| D | D | Acetone/Water | NA | ~Amorphous | 4 (Table 3) |
| E | E | IPA/Water | NA | ~80 | 7 (Table 3) |
| F | F | EtOH at 50° C. | NA | ~83 | J |
| G | G | EtOH at Rm | NA | ~130 | K |

### Example 6

### Characterization of Polymorph Form B

6.1 Thermal Behavior

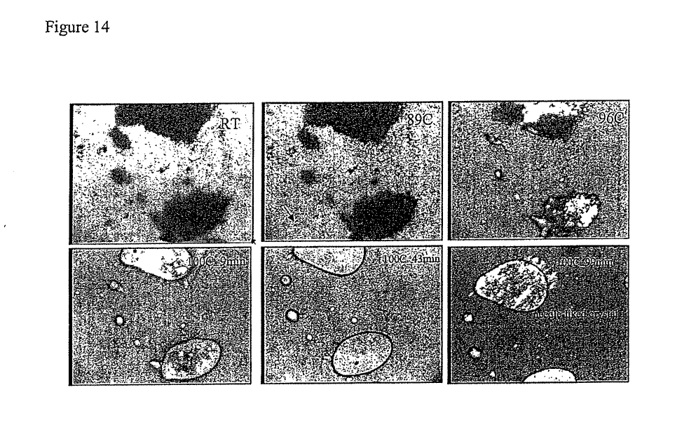

The monohydrate polymorph Form B starts to dehydrate around 83° C. by DSC and TGA (thermal gravimetric) analysis. It is completely dehydrated by ˜110° C. As shown by hot stage microscopy, the material dehydrates with collapse of the crystal lattice starting at 89° C., and re-crystallizes as needle-like crystals around 100° C. (FIG. 14).

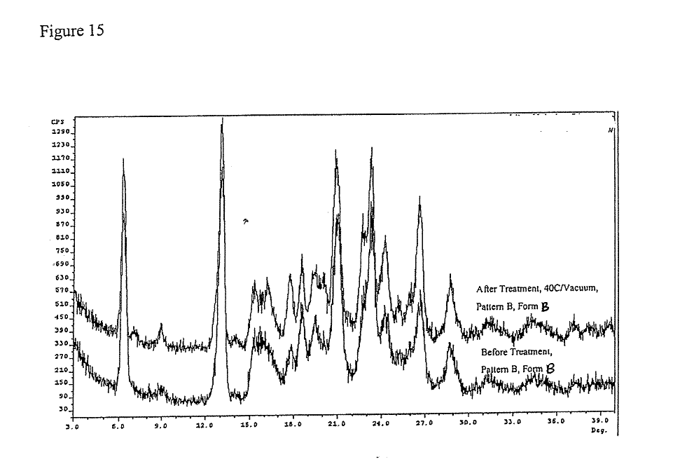

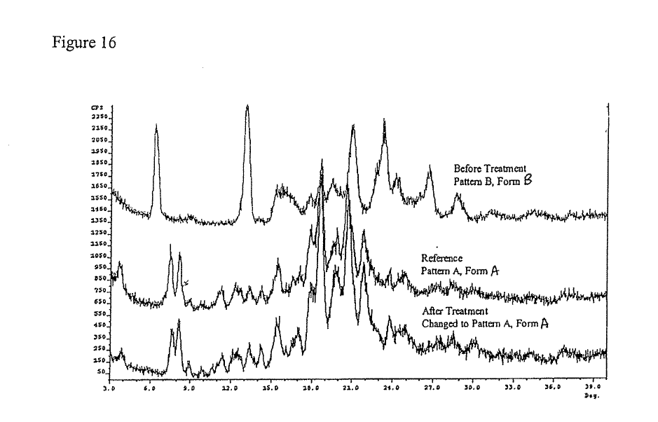

Polymorph Form B (Sample F) was stressed with vacuum drying at 40° C. After 36 hours, the monohydrate did not convert to the anhydrate, retaining 2.95% water and the monohydrate XRD pattern (see FIG. 15). The monohydrate did convert completely to the anhydrate after 23 hours in a 100° C. oven (FIG. 16).

It is concluded that the monohydrate (polymorph Form B) can be produced from the sesquihydrate (polymorph Form C) using a vacuum oven and has sufficient heat stability for at least 3 months at 40° C./75% RH (relative humidity). Conversion to the anhydrate will not occur until about 80° C.

6.2 Hygroscopicity Studies

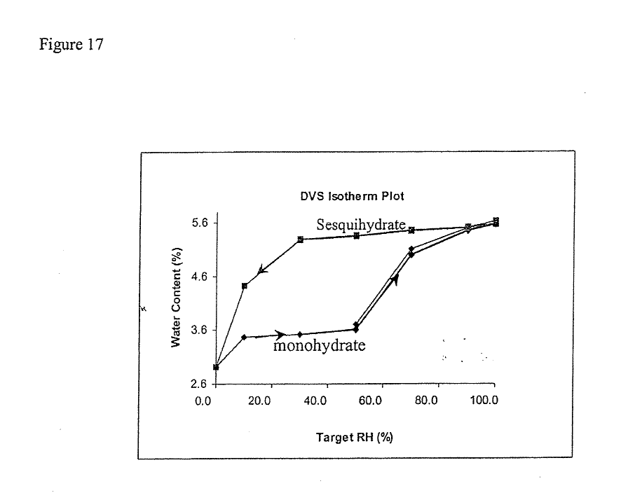

Sample F was subject to Dynamic Vapor Sorption (DVS) analysis at room temperature. The sample was first analyzed by Karl Fischer (KF) titration containing initial moisture content of 3.7% for the monohydrate form. RH cycling started at 50% relative humidity (RH) to 100% RH and down to 0% RH and then back up to 100% RH. A 3-hour/step equilibration period was found insufficient, so the run was repeated with a 6-hour period, which was still not sufficient. The DVS moisture sorption isotherm using the 6-hour equilibration period is presented in FIG. 17.

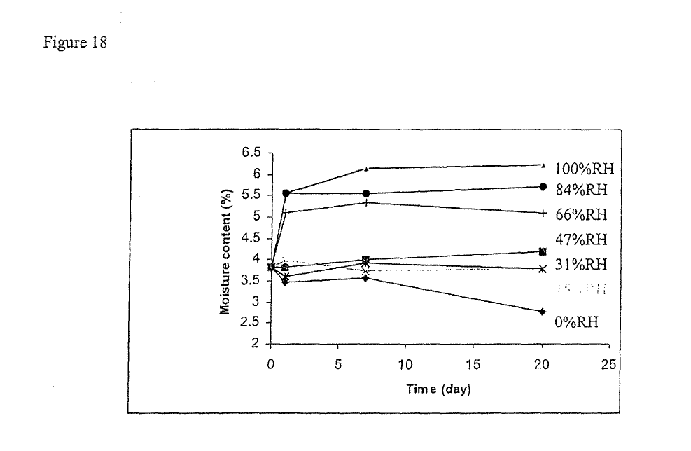

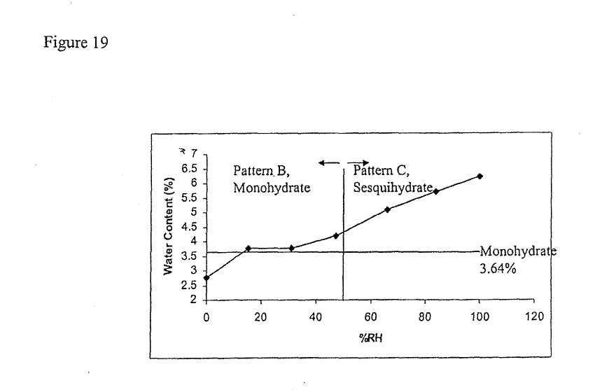

The DVS scan showed hysteresis with two relatively stable Forms: the Form B monohydrate, stable from 0-50% RH and the Form C sesquihydrate, stable from 30-100% RH. As a result, an equilibrium moisture study using desiccators containing saturated salt solutions for humidity control was performed. Sample F was incubated at 0%, 15%_{,} 31%, 66%, 87%, and 100% RH in room temperature for up to 20 days. Samples were periodically assayed by KF titration to give the kinetic profiles. XRD analysis was performed at the end of 20 days. The data is summarized in Table 8 with kinetic and equilibrium data plotted in FIGS. 18 and 19.

TABLE 8
Water Content of Polymorph Form B at Various % RH

| % | Moisture (%) | | | | | | XRD |
|---|---|---|---|---|---|---|---|
| RH | T = 0 | 1-day | 3-day | 4-day | 7-day | 20-day | Polymorph |
| 0 | 3.82 | 3.48 | 3.55 | na | 3.57 | 2.78 | Pattern B, Form B |
| 15 | 3.82 | 3.97 | na | 3.99 | 3.74 | 3.79 | Pattern B, Form B |
| 31 | 3.82 | 3.61 | na | 5.01 | 3.93 | 3.79 | Pattern B, Form B |
| 47 | 3.82 | 3.81 | 3.73 | na | 4.00 | 4.19 | Pattern B, Form B |
| 66 | 3.82 | 5.12 | na | 5.28 | 5.34 | 5.1 | Pattern C, Form C |
| 84 | 3.82 | 5.55 | 5.43 | na | 5.55 | 5.71 | Pattern C, Form C |
| 100 | 3.82 | 5.55 | na | 8.08 | 6.14 | 6.22 | Pattern C, Form C |

XRD data indicated the presence of the monohydrate at 0-47% RH and the sesquihydrate at 66-100% RH, which is consistent with DVS data. The kinetic data showed significant moisture pickup at 66-100% RH within the first day. The monohydrate did lose some water after 20 days at 0% RH (2.78%) and gained some water at 47% RH (4.19%). There was no moisture content plateau for the sesquihydrate.

6.3 Solid State Stability

Sample D containing anhydrous polymorph Form A demonstrated chemical stability after 14-day storage at room temperature, in temperatures up to 80° C., and in a light box (510 fc). Sample F containing polymorph Form B was further investigated at 40° C./75% RH using open and closed vials for 3 months. The monohydrate was also found to be chemically stable, but exhibited polymorphic conversion from Form B to Form C, as shown by DSC and XRD analysis, after 1-month storage at 40° C./75% RH. Water content after 1-month storage at 40° C./75% RH also increased to 5.4%. Therefore, the monohydrate needs to be stored in hermetically sealed containers in order to prevent conversion to the sesquihydrate. Data collected after 2 months of storage at 40° C./75% RH is summarized in Table 9.

TABLE 9
Solid State Stability of Polymorph Form B

| | | HPLC Impurity | KF | XRD |
|---|---|---|---|---|
| Conditions | Time | (%) | (%) | Polymorph |
| Initial | 0 | 0.30 | 3.82 | Form B Pattern B |
| 40 C./75% RH | 4 wk | 0.36 | 5.34 | Form C Pattern C |
| Open | 8 wk | 0.49 | 5.53 | Form C Pattern C |
| 40 C./75% RH | 4 wk | 0.34 | 5.35 | Form C Pattern C |
| Closed | 8 wk | 0.41 | 5.79 | Form C Pattern C |

6.4 Solution Stability

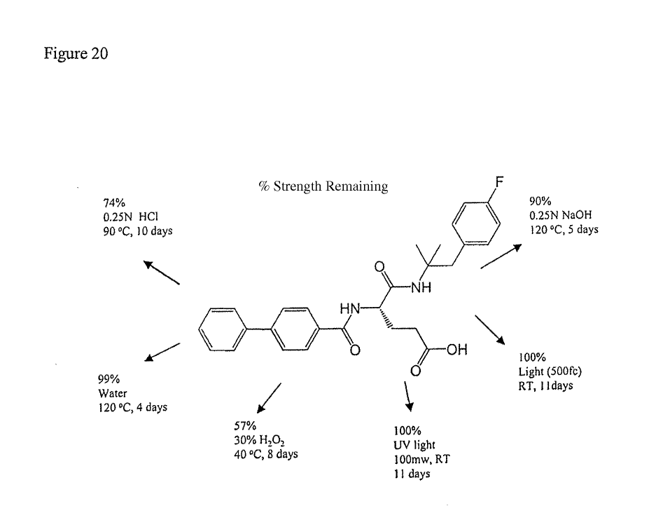

A forced degradation study was performed to generate potential degradation products and assess stability liabilities. N²-(1,1′-biphenyl-4-ylcarbonyl)-N¹-[2-(4-fluorophenyl)-1,1-dimethylethyl]-L-α-glutamine was solubilized at 387 μg/mL with 25% acetonitrile in acid, base, peroxide and water under heat and light stress conditions. The results indicate that the compound is subject to acid and peroxide degradation. The forced degradation scheme for N²-(1,1′-biphenyl-4-ylcarbonyl)-N¹-[2-(4-fluorophenyl)-1,1-dimethylethyl]-L-α-glutamine is presented in FIG. 20.

6.5 Degradant Identification

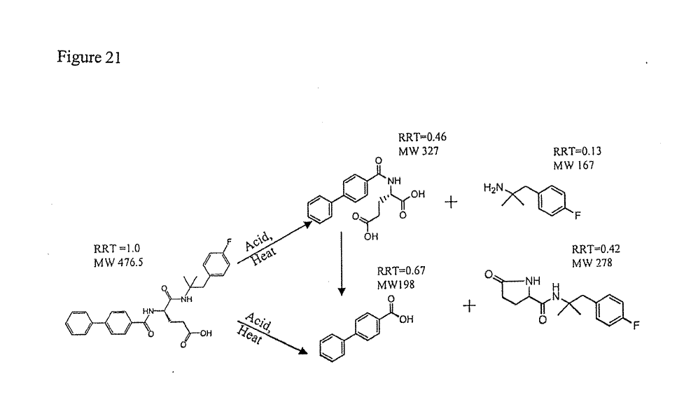

The acid degradation mechanism is hydrolysis of the two amide bonds. Hydrolysis of the first amide bond yields fragments of MW (molecular weight) 167 and MW 327. Hydrolysis of the second amide bond yields fragments of MW 198 and MW 278. The MW 327 degradant can further hydrolyze to a fragment of MW 198. These two degradants (MW 198 and MW327) were isolated by semi-prep HPLC and characterized by LC-MS and NMR. The molecular structures for the two major degradants have been confirmed based on exact mass, NMR, LC-MS fragmentation patterns, and HPLC relative retention times. The proposed degradation pathway is shown in FIG. 21.

6.6 pH Solubility Profile

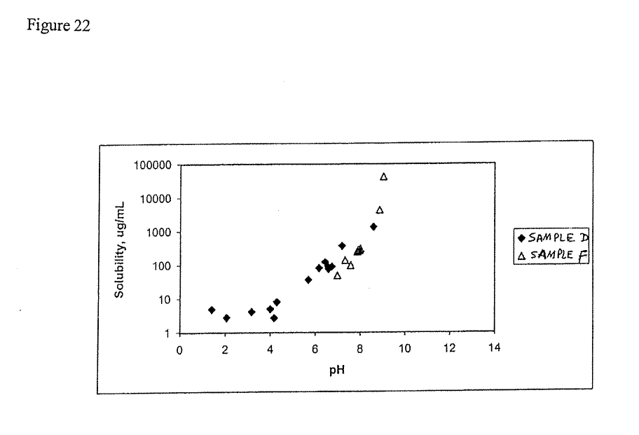

N²-(1,1′-biphenyl-4-ylcarbonyl)-N¹-[2-(4-fluorophenyl)-1,1-dimethylethyl]-L-α-glutamine is an acid with a pKa of 5.3 as measured from a pH solubility profile. The pH solubility profile was generated first with Sample D containing polymorph Form A, and then with Sample F containing polymorph Form B, in HCl/NaOH solutions. After 24 hour equilibration, suspensions were centrifuged and the supernatants assayed by HPLC. The samples were not filtered since there is significant compound loss due to filter binding. The pH-solubility profile is presented FIG. 22.

The solubility at pH<4 is ˜4.5 ug/mL for Sample D but below the HPLC detection limit of 11 ng/mL for Sample F. Above pH 4, solubility increases with increasing pH for both batches. At pH 12.8, the solubility is 4.7 mg/mL, which represents the solubility of the sodium salt.

The solubility data was analyzed as a function of pH using the Henderson-Hasselbach equation:

Solubility=S₀·[10^{(PH-pKa)}+1],

where S₀ is the intrinsic solubility of the free acid. A pKa value of 5.3 was determined via nonlinear regression analysis using the computer program, SigmaPlus. The curve fitting results are shown in Table 10.

TABLE 10
pKa Fitting Results

| Parameter | Value | StdErr | CV (%) | Dependencies |
|---|---|---|---|---|
| S₀ | 4.5 (μg/mL) | 2.5 (μg/mL) | 55.9 | 1.0 |
| pKa | 5.3 | 0.3 | 4.9 | 1.0 |

The measured pKa of 5.3 is lower than the calculated pKa value of 4.4 from the computdrugpka/c program based on the Hammett and Taft equation. It is theorized that the steric shielding from the two aromatic ring systems may reduce the acidity of the carboxylic group.

6.7 Biopharmaceutical Properties

Two CACO-2 studies were conducted to evaluate GI membrane permeability of N²-(1,1′-biphenyl-4-ylcarbonyl)-N¹-[2-(4-fluorophenyl)-1,1-dimethylethyl]-L-α-glutamine. The first study was conducted at pH 7.4 using metoprolol, the FDA high permeability calibration compound (Table 11). A ratio of 0.25 to metoprolol in the apical to basolateral direction (A→B) indicated low permeability. The B→A/A→B ratio of 2.36 suggested slight efflux. Discounting the efflux, the ratio to metoprolol in the opposite direction (B→A) is still low at 0.59, supporting a low permeability classification for the compound.

TABLE 11
Caco-2 Study using Metoprolol as Reference

| | Concentration | Flux | | | Ratio to | Ratio of |
|---|---|---|---|---|---|---|
| Compound | (mg/mL) | Direction | Pₐₚₚ (nm/s) | % Recovery | Metoprolol | B→A/A→B |
| Compound (I) | 0.005 | A → B | 149.1 | 100 | 0.25 | 2.36 |
| | | B → A | 373.7 | 156 | 0.59 | |
| Metoprolol | 0 | A → B | 603.5 | 109 | | |
| | | B → A | 632.1 | 115 | | |
| Atenolol | 0 | A → B | 13. | 106 | | |
| | | B → A | 10.5 | 100 | | |
| DigoxinS | 0.003905 | A → B | 20.6 | 117 | | |
| | | B → A | 294.3 | 94 | | |

The second CACO-2 study gave somewhat different results (Table 12). Because metoprolol interferes with the LC/MS assay, propranolol, which has a permeability that is not as high as metoprolol, was used as the FDA calibration compound. The apical pH was controlled at 6 and basolateral pH at pH 7. In addition, verapamil was used as a competitive inhibitor of the Pgp (P-glycoprotein) transporter. The ratios to propranolol were 1.00 and 0.77 with and without verapamil. Since verapamil did not enhance compound permeability, Pgp is not involved. The two values were averaged to give a mean ratio of 0.88, indicating low permeability. The B→A/A→B ratios were low at 0.3 and 0.3, with and without verapamil, respectively, indicating again no efflux but active transport.

TABLE 12
Caco-2 study using Propranolol as Reference

| | Concentration | Flux | | Ratio to | Ratio of |
|---|---|---|---|---|---|
| Compound | (mg/mL) | Direction | Pₐₚₚ (nm/s) | Propranolol | B→A/A→B |
| Compound (I) | 0.025 | A → B | 624 | 1.00 | 0.3 |
| | 0.025 | B → A | 185 | | |
| Compound (I) | 0.025 | A → B | 483 | 0.77 | 0.3 |
| (plus Verapamil) | 0.025 | B → A | 162 | | |
| Propranolol | | A → B | 626 | | |

N²-(1,1′-biphenyl-4-ylcarbonyl)-N¹-[2-(4-fluorophenyl)-1,1-dimethylethyl]-L-α-glutamine showed high permeability in the in situ rat perfusion model. The permeability was 18.2 nm/sec, which is higher than the 14.5 nm/sec value obtained for metoprolol (Table 13).

TABLE 13
Rat Perfusion study using Metoprolol as Reference

| | Metoprolol | Compound (I) | |
|---|---|---|---|
| | 20 μg/mL | 20 μg/mL | Ratio to Metoprolol |
| Peff × 10⁻¹ (μm/sec) | 1.45 | 1.82 | 1.26 |

6.8 Animal Formulation

The solubility of N²-(1,1′-biphenyl-4-ylcarbonyl)-N¹-[2-(4-fluorophenyl)-1,1-dimethylethyl]-L-α-glutamine in water is below 0.1 mg/mL with a resulting pH around 5.5. Solubility is enhanced slightly in the 2% Tween/0.5% methylcellulose toxicology vehicle, ranging from 0.47 to 0.22 mg/mL. 2% Tween may also increase GI membrane permeability. Oral bioavailability (F %) from the Tox suspensions are 29% and ˜100% at 25 mg/kg in rat and dog, respectively.

In the rat, the bioavailability increased from 29% (25 mg/kg) to near 100% (100 mg/kg) suggesting saturation of first pass metabolism. In the dog, bioavailability was lowered from ˜100% to 65% when a capsule containing neat drug was used. A prolonged absorption phase was observed together with decreasing bioavailability with increasing dose in both animal species. These results suggest dissolution rate-limited absorption at toxicological doses. However, the absorption at pharmacological doses seemed reasonably complete albeit with delayed Tₘₐₓ at lower GI when the drug is more soluble.

### Example 7

### Preparation of Polymorph Form C

Polymorph Form C was formed by crystallization from a slurry in ethanol and water.

### Example 8

### Characterization of Polymorph Form C

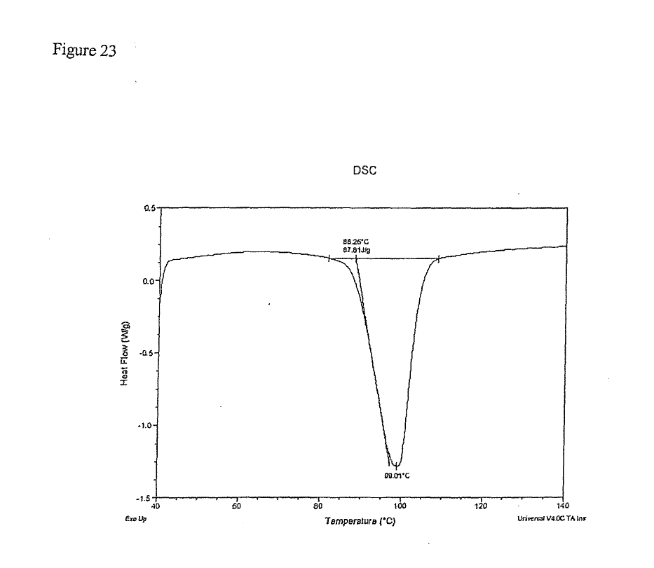

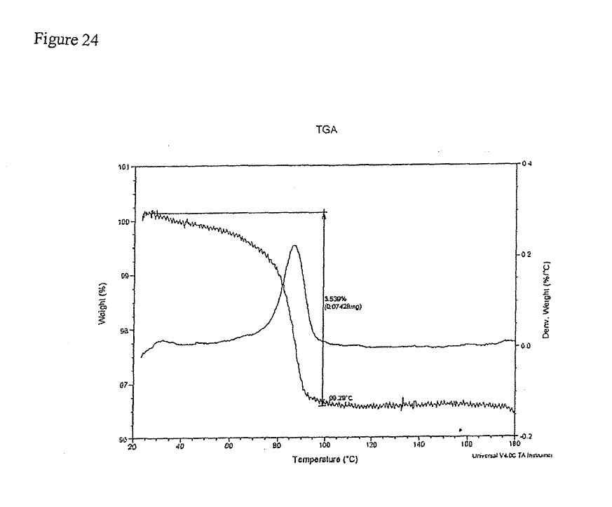

DSC and TGA (thermal gravimetric analysis) thermograms of polymorph Form C are shown in FIGS. 23 and 24, respectively.

### Example 9

### Preparation of Polymorph Form D

Polymorph Form D was formed by crystallization from a slurry in acetone and water.

### Example 10

### Preparation of Polymorph Form E

Polymorph Form E was formed by crystallization from a slurry in isopropyl alcohol (IPA) and water.

### Example 11

### Preparation of Polymorph Form F

Polymorph Form F was formed by crystallization from a slurry in ethanol at about 50° C.

### Example 12

### Preparation of Polymorph Form G

Polymorph Form G was formed by crystallization from a slurry in ethanol and water at about 20-25° C.

### Example 13

### Solubility of N²-(1,1′-biphenyl-4-ylcarbonyl)-N¹-[2-(4-fluorophenyl)-1,1-dimethylethyl]-L-α-glutamine

The solubility of compound (I) in various solvents at 25° C. and 50° C. is shown in Table 14.

TABLE 14

| Solubility (mg/ml) | 25° C. | 50° C. |
|---|---|---|
| Toluene | 4.3 | 15.6 |
| Heptane | 0.7 | −0.3 |
| EtAc (ethyl acetate) | >109 | |
| Isopropyl acetate | 76.1 | >123.1 |
| (IPAc) | | |
| Isopropyl alcohol | >122 | |
| (IPA) | | |
| Acetonitrile | 43.0 | >132.6 |
| Water | 1.6 | 1.8 |
| Ethanol | >137 | |

### Example 14

### Polymorphism screening of N²-(1,1′-biphenyl-4-ylcarbonyl)-N¹-[2-(4-fluorophenyl)-1,1-dimethylethyl]-L-α-glutamine

Two parallel approaches were taken to assess polymorphism. The first approach was aging slurries of the compound in solvents or mixture of solvents at room temperature and 50° C. for 6-8 days to promote formation of more stable crystal forms that might exist. The second approach was re-crystallization of the compound in different solvents by using cooling, anti-solvent addition and evaporation or a combination of any two steps as super-saturation generation methods. The solids from each experiment were dried overnight under full vacuum at 40° C. Details of experiments and observations are shown in the following tables:

Cooling crystallization: Table 15;

Anti-solvent addition crystallization: Table 16;

Cooling followed by anti-solvent addition crystallization: Table 17;

Anti-solvent addition followed by cooling crystallization: Table 18;

Slurry experiments: Table 19; and

Slurry followed by anti-solvent addition: Table 20.

TABLE 15
Cooling crystallization

| | | Amount of | | |
|---|---|---|---|---|
| | | compound (I) and | DSC | XRD pattern compared |
| Sample | Solvent | solvent | onset (° C.) | with original |
| 1 | Toluene | 96.7 mg in 1 ml toluene | 135 | Almost the same except one |
| | | | | missing small peak (13.47) |
| 2 | IPAc | 152.7 mg in 1 ml IPA | 135 | Almost the same except three |
| | | | | missing small peaks |
| 3 | Acetonitrile | 148.8 mg in 1 ml | 133 | Almost the same except one |
| | | acetonitrile | | missing small peak |
| 4 | IPA | 184.4 mg in 1 ml IPA | 80, 88 | Different (I), but very low |
| | | | | crysallinity |
| 5 | Ethanol | 265.0 mg in 1 ml ethanol | 79 | Different (I), but lower crysallinity |
| 6 | Ethanol | 167.7 mg in 1 ml ethanol | 80 | Different (I), but very low |
| | | | | crysallinity |
| 7 | Ethanol | 167.0 mg in 1 ml ethanol | 81, 137 | Different and low crystallinity |
| 8 | Acetonitrile | 132.6 mg in 1 ml | 136 | The same as original |
| | | acetonitrile | | |

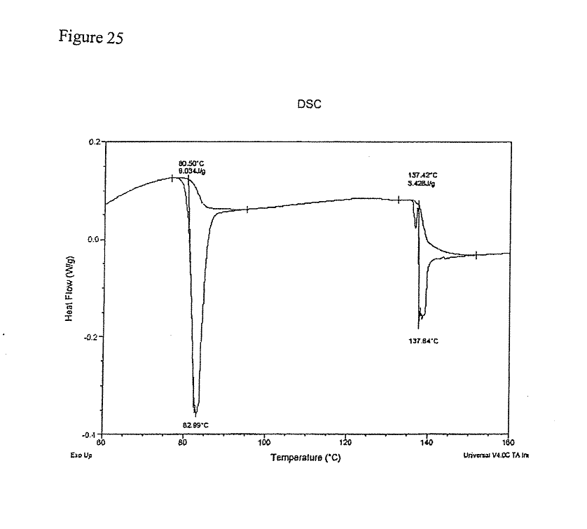

Sample 7 was prepared by slow cooling in an attempt to increase the crystallinity. However, the solution did not crystallize even cooled to room temperature. Crystallization occurred after stirring for overnight. The final product contained mostly low crystalline material with a very small amount of higher melting point form which is shown in the DSC profile (FIG. 25).

TABLE 16
Anti-solvent addition crystallization

| | | Amount of | | |
|---|---|---|---|---|
| | | compound (I) | DSC | XRD pattern compared |
| Sample | Solvents | and solvents | onset (° C.) | with original |
| 9 | IPA + water | 131.5 mg in 1 ml | 79 | Different (I), but lower crysallinity, |
| | | IPA | | extra peak at 15.2 |
| 10 | Acetone + | 9.8 mg in 1 ml | 136 | Almost the same except missing |
| | water | acetone | | small peak |
| 11 | IPA + water | 99.1 mg in 1 ml | 78, and 88 | Different (I) reasonable |
| | | IPA | | crystallinity, extra peak at 23.3 |
| 12 | Ethyl | 109.0 mg in 1 ml | 137 | Same |
| | acetate + | ethyl acetate | | |
| | heptane | | | |
| 13 | IPAc + | 100.5 mg in 1 ml | 137 | Almost the same except two |
| | heptane | IPAc | | missing small peaks |

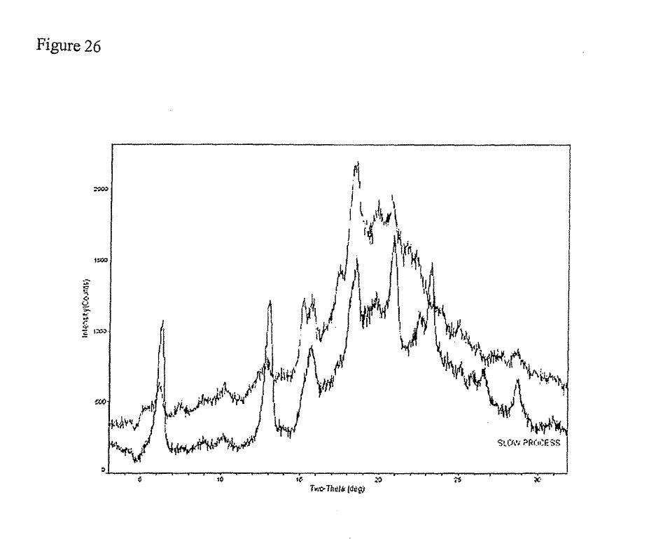

Sample 11 was prepared by a process of slow anti-solvent addition in an attempt to increase crystallinity. Sample 11 did show improved crystallinity over fast water addition, as shown with Sample 9. XRD patterns of the two Samples are shown in FIG. 26.

TABLE 17
Cooling followed by anti-solvent addition crystallization

| | | Amount of | DSC | |
|---|---|---|---|---|
| | | compound (I) | onset | XRD pattern compared |
| Sample | Solvents | and solvents | (° C.) | with original |
| 14 | Ethanol + | 177.5 mg in | 79 | Different (I), but lower |
| | water | 0.8 ml | | crysallinity |
| | | ethanol | | |
| 15 | IPAc + | 140.1 mg in | 137 | Almost the same except |
| | heptane | 0.8 ml | | some missing and extra |
| | | IPAc | | small peaks, higher |
| | | | | crystallinity |

TABLE 18
Anti-solvent addition followed by cooling crystallization

| | | Amount of | DSC | XRD pattern |
|---|---|---|---|---|
| | | compound (I) | onset | compared with |
| Sample | Solvents | and solvents | (° C.) | original |
| 16 | Ethanol + | 110.4 mg in 1 ml | 78, and 86 | Different (I), but |
| | water | ethanol | | reasonable |
| | | | | crystallilinity, |
| | | | | extra peaks at 21.0, |
| | | | | 23.4, 26.8 |

TABLE 19
Slurry experiments

| | | DSC | XRD pattern |
|---|---|---|---|
| | | onset | compared with |
| Sample | Solvents | (° C.) | original |
| 17 | Acetonitrile | 137 | The same but |
| | | | higher crystallinity |
| 18 | Toluene | 134 | Same |
| 19 | IPAc | 140 | Same |
| 20 | Tert-butyl methyl | 133 | Same |
| | ether (tBME) | | |
| 21 | Mixture of | 83 | Different (II), but higher |
| | ethanol + water | | crystallinity |
| 22 | tBME at 50° C. | 139 | Similar with several |
| | | | missing and extra |
| | | | peaks |
| 23 | Ethanol | 84 | Different (III), but higher |
| | | | crystallinity |
| 24 | Ethanol | 80 | Different (I), but lower |
| | | | crysallinity, with |
| | | | missing peak at 9.0 |
| 25 | Ethanol at 50° C. | 82 | Different (IV), but |
| | | | higher crystallinity |

TABLE 20
Slurry followed by anti-solvent addition

| | | Amount of | DSC | XRD pattern |
|---|---|---|---|---|
| | | compound (I) | onset | compared with |
| Sample | Solvents | and solvents | (° C.) | original |
| 26 | Ethanol + | 118.7 mg in 1.0 ml | 79 | Different, but very |
| | water | ethanol | | low crysallinity |
| 27 | Ethanol + | 137.0 mg in 1.0 ml | 80, 134 | Different, but lower |
| | water | ethanol | | crysallinity |

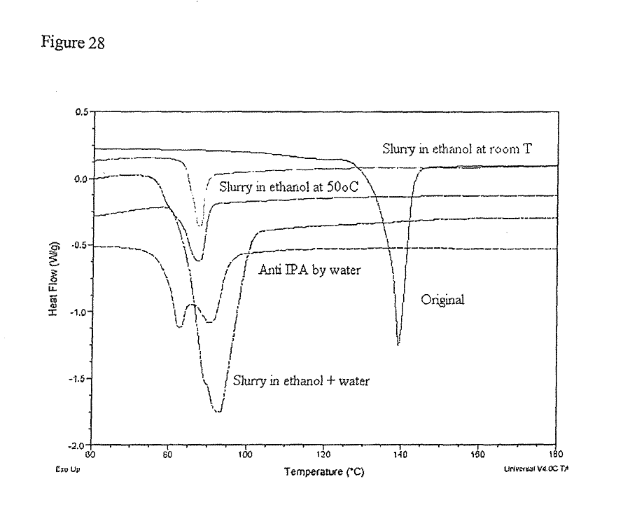

Most crystallization operations from alcohols (e.g., IPA and ethanol) would result in different forms of compound (I) but with very low crystallinity. The product prepared from a process of slow crystallization by anti-solvent addition of IPA in water showed reasonable crystallinity, as shown in FIG. 27. This form is very close to the form obtained with a slurry of compound (I) in a mixture of ethanol and water at room temperature. A slurry in ethanol at room temperature, in ethanol at 50° C., and in a mixture of water and ethanol all resulted in forms with higher crystallinity but lower melting point compared to the original compound. DSC thermograms of these different polymorphs are shown in FIG. 28.

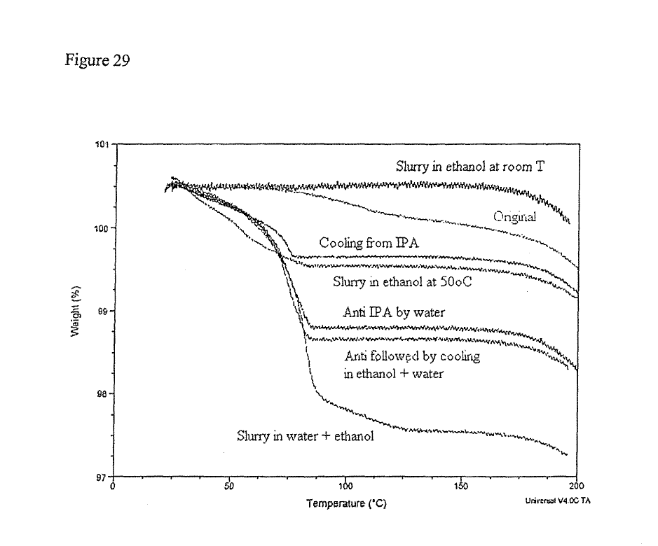

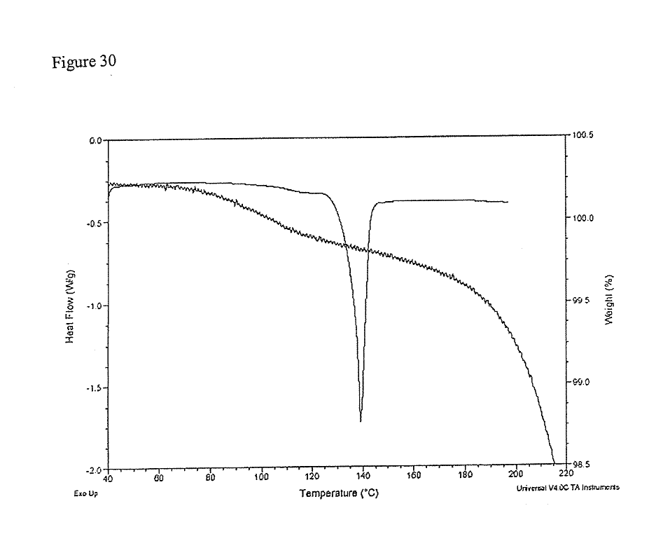

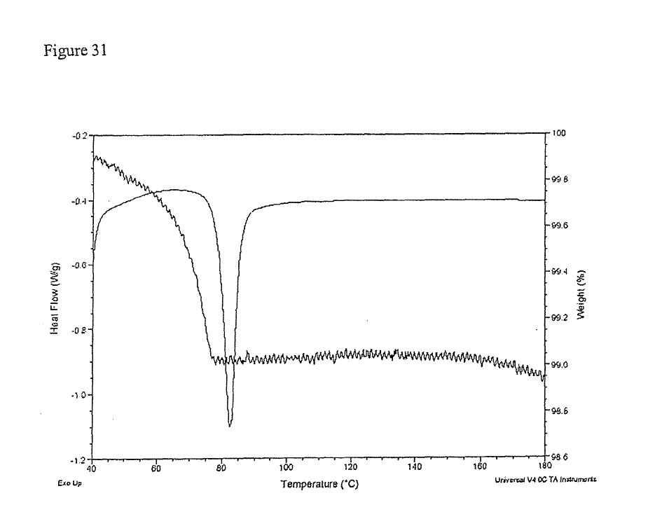

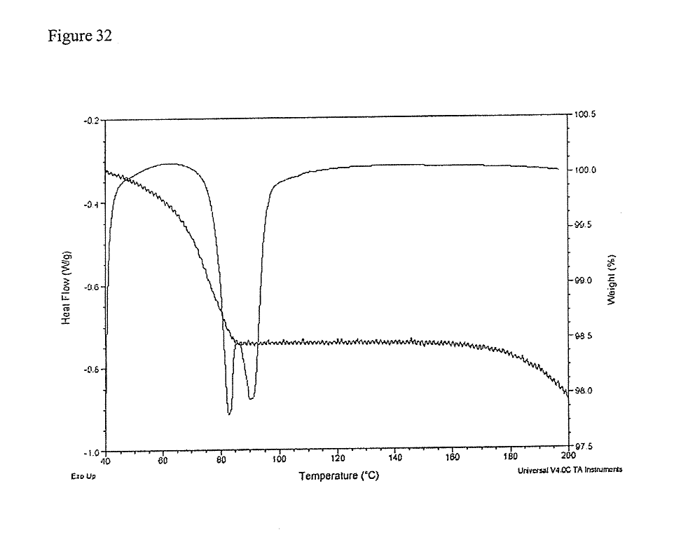

Two peaks were observed in the product obtained from the process of slow crystallization process by anti-solvent addition of IPA and water, and a shoulder was observed in the DSC thermogram of the product prepared from a slurry in ethanol at 50° C. TGA (thermal gravimetric analysis) was used to check if the extra peak or shoulder in the DSC event was a result of de-solvation. TGA data obtained for various samples are shown in Table 21 and TGA profiles of the various polymorphs are shown in FIG. 29. TGA and DSC profiles for the original compound, the product prepared by cooling followed by anti-solvent addition in ethanol and water, and the product prepared by anti-solvent addition of IPA in water are shown together in FIGS. 30, 31, and 32, respectively. Samples prepared from anti-solvent addition of IPA and water (Sample 11), a slurry of ethanol and water (Sample 21), and a slurry in ethanol at 50° C. (Sample 25) were analyzed by GC (gas chromatography) to detect residue solvent to determine the cause of weight loss observed in the TGA experiments. There was no detectable amount of solvent found in all three samples.

TABLE 21
TGA data for various samples

| Sample | Description | Wt % | T range |
|---|---|---|---|
| Original | Original compound | 0.4% | 58° C. to 131° C. |
| compound | | | |
| 16 | Anti-solvent addition followed by | 1.8% | 25° C. to 86° C. |
| | cooling in ethanol and water | | |
| 14 | Cooling followed by anti-solvent | 1.0% | 25° C. to 77° C. |
| | addition in ethanol and water | | |
| 11 | Anti-solvent addition of IPA | 1.7% | 25° C. to 86° C. |
| | and water | | |
| 4 | Cooling crystallization from IPA | 0.9% | 25° C. to 78° C. |
| 21 | Slurry in ethanol + water | 2.6% | 25° C. to 90° C. |
| 25 | Slurry in ethanol at 50° C. | 1.1% | 25° C. to 84° C. |
| 23 | Slurry in ethanol at room | 0% | Before 160° C. |
| | temperature | | |

### Example 15

### Preparation of Polymorph Pseudo Form A

Polymorph pseudo Form A was formed by dissolving the starting material compound (I) in an organic solvent to form a solution and evaporating the organic solvent to form the crystalline polymorph. The organic solvent can be a mixture of ethyl acetate (EtAc) and heptane; toluene; isopropyl acetate (IPAc); acetonitrile; a mixture of acetone and water; tert-butyl methyl ether; or a mixture of isopropyl acetate and heptane.

### Example 16

### Characterization of Polymorph Pseudo Form A

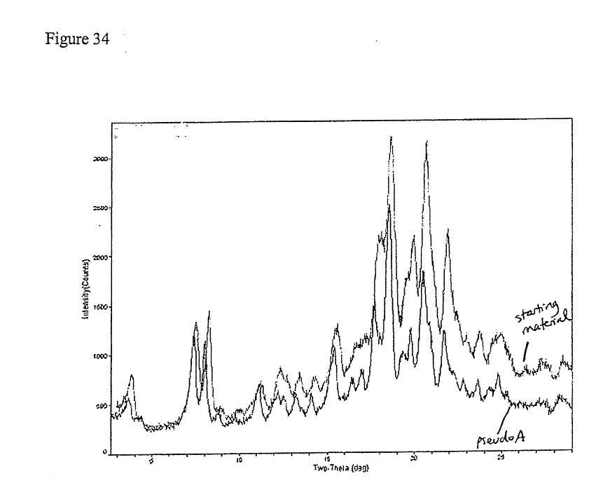

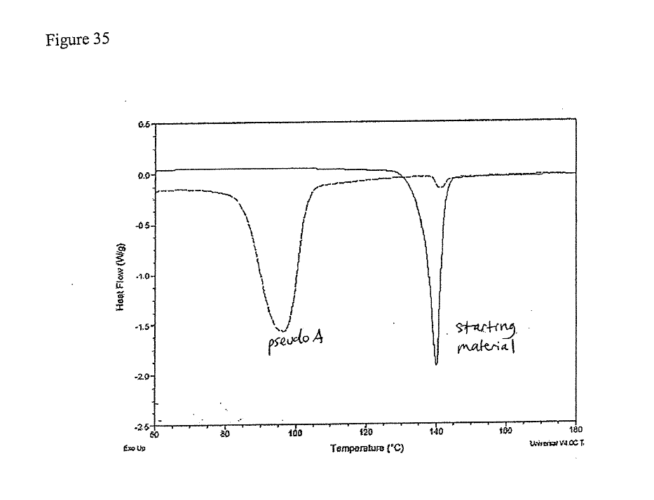

Changes to the powder XRD pattern or melting point (136° C.) were not observed for a batch of the starting material N²-(1,1′-biphenyl-4-ylcarbonyl)-N¹-[2-(4-fluorophenyl)-1,1-dimethylethyl]-L-α-glutamine equilibrated in a slurry in water at 50° C. for 12-24 hr. In comparison, most of Sample E, containing polymorph pseudo Form A, transformed to a lower melting point polymorph form in water at 50° C. after 12-24 hr. Powder XRD patterns comparing the starting material to polymorph pseudo Form A are shown in FIG. 34, and DSC thermograms of both batches at 50° C. are shown in FIG. 35.

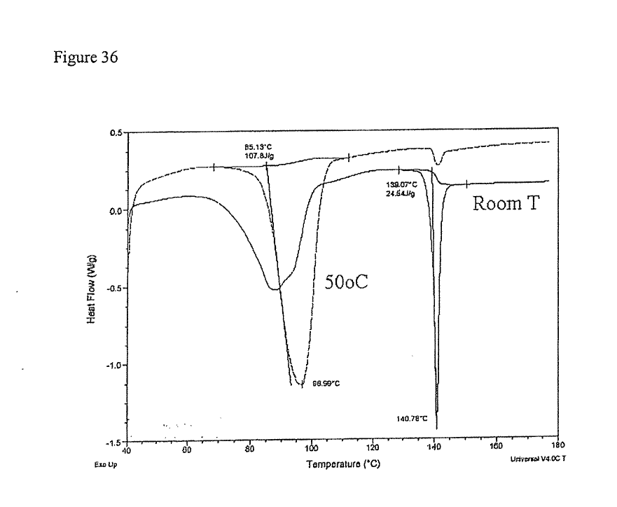

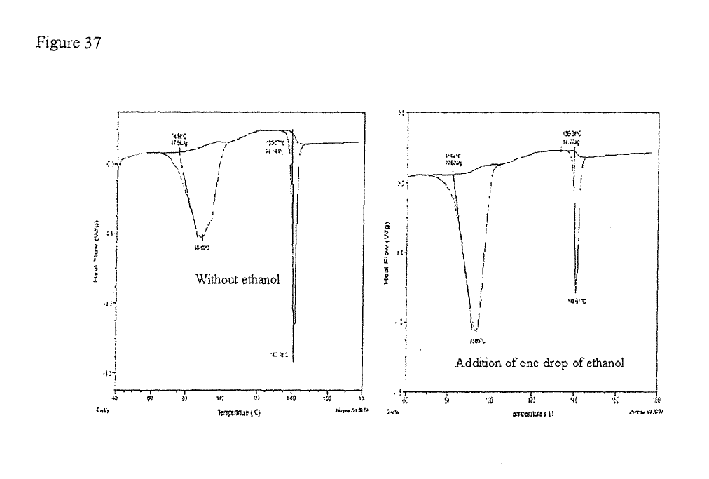

The transformation rate of polymorph pseudo Form A in water was found to be dependent on temperature, and alternatively, on the addition of ethanol. Higher temperature expedited the transformation of pseudo Form A to a lower melting form (FIG. 36). The addition of only one drop of ethanol (10 μL) also increased the rate of transformation (FIG. 37).

What is claimed is:

1. A polymorph of N²-(1,1′-biphenyl-4-ylcarbonyl)-N¹-[2-(4-fluorophenyl)-1,1-dimethylethyl]-L-α-glutamine, wherein the polymorph is Form A, Form B, Form C, Form D, Form E, Form F, Form G or pseudo Form A.

2. The polymorph of claim 1, wherein the polymorph is Form A.

3. The polymorph of claim 2, wherein the polymorph has a powder X-ray diffraction pattern comprising peaks at diffraction angles (degrees 2θ) of about 7.45, 8.01, 15.40, 17.67, 18.49, 19.71 and 20.44.

4. The polymorph of claim 2, wherein the polymorph has a DSC extrapolated melting temperature onset of about 134° C.

5. The polymorph of claim 2 having a powder X-ray diffraction pattern substantially as shown in FIG. 1.

6. The polymorph of any of claims 2-5, wherein the polymorph is a substantially pure polymorph of Form A.

7. The polymorph of claim 1, wherein the polymorph is Form B.

8. The polymorph of claim 7, wherein the polymorph has a powder X-ray diffraction pattern comprising peaks at diffraction angles (degrees 2θ) of about 6.32, 13.12, 21.01, 23.36, 24.23 and 26.02.

9. The polymorph of claim 7, wherein the polymorph has a DSC extrapolated melting temperature onset of about 83° C.

10. The polymorph of claim 7 having a powder X-ray diffraction pattern substantially as shown in FIG. 2.

11. The polymorph of any of claims 7-10, wherein said polymorph comprises about 5% or less by weight water.

12. The polymorph of any of claims 7-11, wherein the polymorph is a substantially pure polymorph of Form B.

13. The polymorph of claim 1, wherein the polymorph is Form C.

14. The polymorph of claim 13, wherein the polymorph has a powder X-ray diffraction pattern comprising peaks at diffraction angles (degrees 2θ) of about 6.41, 12.54, 14.34, 16.90, 17.80, 19.16, 23.93, 25.40 and 26.52.

15. The polymorph of claim 13, wherein the polymorph has a DSC extrapolated melting temperature onset of about 83-89° C.

16. The polymorph of claim 13 having a powder X-ray diffraction pattern substantially as shown in FIG. 3.

17. The polymorph of any of claims 13-16, wherein the polymorph is a sesquihydrate of polymorph Form B, containing about 1.5 mol of water per mol of the polymorph of Form B.

18. The polymorph of any of claims 13-17, wherein the polymorph is a substantially pure polymorph of Form C.

19. The polymorph of claim 1, wherein the polymorph is Form D.

20. A method for the preparation of the polymorph of claim 19, which comprises equilibrating a slurry of N²-(1,1′-biphenyl-4-ylcarbonyl)-N¹-[2-(4-fluorophenyl)-1,1-dimethylethyl]-L-α-glutamine in acetone and water, and isolating the polymorph defined in claim 19.

21. The polymorph Form D of N²-(1,1′-biphenyl-4-ylcarbonyl)-N¹-[2-(4-fluorophenyl)-1,1-dimethylethyl]-L-α-glutamine prepared by the method of claim 20.

22. The polymorph of claim 19 or 21, wherein the polymorph is a substantially pure polymorph of Form D.

23. The polymorph of claim 1, wherein the polymorph is Form E.

24. The polymorph of claim 23, wherein the polymorph has a powder X-ray diffraction pattern comprising peaks at diffraction angles (degrees 2θ) of about 6.44, 12.59, 18.54, 19.09, 22.04 and 25.57.

25. The polymorph of claim 23, wherein the polymorph has a DSC extrapolated melting temperature onset of about 80° C.

26. The polymorph of claim 23 having a powder X-ray diffraction pattern substantially as shown in FIG. 4.

27. The polymorph of any of claims 23-26, wherein the polymorph is a substantially pure polymorph of Form E.

28. The polymorph of claim 1, wherein the polymorph is Form F.

29. The polymorph of claim 28, wherein the polymorph has a powder X-ray diffraction pattern comprising peaks at diffraction angles (degrees 2θ) of about 5.80, 6.24, 17.84, 18.50, 20.42 and 20.76.

30. The polymorph of claim 28, wherein the polymorph has a DSC extrapolated melting temperature onset of about 83° C.

31. The polymorph of claim 28 having a powder X-ray diffraction pattern substantially as shown in the first trace from the top of FIG. 27.

32. The polymorph of any of claims 28-31, wherein the polymorph is a substantially pure polymorph of Form F.

33. The polymorph of claim 1, wherein the polymorph is Form G.

34. The polymorph of claim 33, wherein the polymorph has a powder X-ray diffraction pattern comprising peaks at diffraction angles (degrees 2θ) of about 5.90, 11.50, 13.16, 17.84, 20.20, 21.20, 22.50, and 26.70.

35. The polymorph of claim 33, wherein the polymorph has a DSC extrapolated melting temperature onset of about 83° C.

36. The polymorph of claim 33 having a powder X-ray diffraction pattern substantially as shown in the second trace from the top of FIG. 27.

37. The polymorph of any of claims 33-36, wherein the polymorph is a substantially pure polymorph of Form G.

38. The polymorph of claim 1, wherein the polymorph is pseudo Form A.

39. The polymorph of claim 38, wherein the polymorph has a powder X-ray diffraction pattern comprising peaks at diffraction angles (degrees 2θ) of about 7.45, 8.01, 15.17, 17.67, 18.49, 19.71 and 20.44.

40. The polymorph of claim 38, wherein the polymorph has a DSC extrapolated melting temperature onset of about 138° C.

41. The polymorph of claim 38 having a powder X-ray diffraction pattern substantially as shown in FIG. 5.

42. The polymorph of any of claims 38-41, wherein the polymorph is a substantially pure polymorph of pseudo Form A.

43. A composition comprising the polymorph of any of claims 1-5, 7-11, 13-17, 19-21, 23-26, 28-31, 33-36 or 38-41 and a pharmaceutically acceptable carrier.

44. The composition of claim 43, wherein at least 50% by weight of the total of N²-(1,1′-biphenyl-4-ylcarbonyl)-N¹-[2-(4-fluorophenyl)-1,1-dimethylethyl]-L-α-glutamine in said composition is present as said polymorph.

45. The composition of claim 43, wherein at least 70% by weight of the total of N²-(1,1′-biphenyl-4-ylcarbonyl)-N¹-[2-(4-fluorophenyl)-1,1-dimethylethyl]-L-α-glutamine in said composition is present as said polymorph.

46. The composition of claim 43, wherein at least 80% by weight of the total of N²-(1,1′-biphenyl-4-ylcarbonyl)-N¹-[2-(4-fluorophenyl)-1,1-dimethylethyl]-L-α-glutamine in said composition is present as said polymorph.

47. The composition of claim 43, wherein at least 90% by weight of the total of N²-(1,1′-biphenyl-4-ylcarbonyl)-N¹-[2-(4-fluorophenyl)-1,1-dimethylethyl]-L-α-glutamine in said composition is present as said polymorph.

48. The polymorph of any of claim 6, 12, 18, 22, 27, 32, 37 or 42, wherein the polymorph contains less than 10% by weight of impurities.

49. The polymorph of claim 48, wherein the polymorph contains less than 5% by weight of impurities.

50. The polymorph of claim 48, wherein the polymorph contains less than 1% by weight of impurities.

51. A composition comprising the polymorph of any of claims 6, 12, 18, 22, 27, 32, 37, 42 or 48-50 and a pharmaceutically acceptable carrier.

52. A composition consisting essentially of the polymorph of any of claims 1-42 or 48-50 and a pharmaceutically acceptable carrier.

53. The composition of any of claims 43-47 or 51-52, wherein the pharmaceutically acceptable carrier is suitable for oral administration and the composition comprises an oral dosage form.

54. A method of inhibiting the activity of a metalloproteinase, in a mammal in need thereof, which comprises, administering to the mammal an effective dose of the composition of any of claims 43-47 or 51-53.

55. The method of claim 54, wherein the metalloproteinase is a matrix metalloproteinase or an aggrecanase.

56. The method of claim 55, wherein the aggrecanase is aggrecanase-1 or aggrecanase-2.

57. A method for treating a metalloproteinase-related disorder, in a mammal in need thereof, which comprises, administering to the mammal an effective dose of the composition of any of claims 43-47 or 51-53.

58. The method of claim 57, wherein the metalloproteinase is a matrix metalloproteinase or an aggrecanase.

59. The method of claim 58, wherein the aggrecanase is aggrecanase-1 or aggrecanase-2.

60. The method of claim 57, wherein the metalloproteinase-related disorder is selected from arthritic disorders, osteoarthritis, cancer, rheumatoid arthritis, asthma, chronic obstructive pulmonary disease, atherosclerosis, age-related macular degeneration, myocardial infarction, corneal ulceration and other ocular surface diseases, hepatitis, aortic aneurysms, tendonitis, central nervous system diseases, abnormal wound healing, angiogenesis, restenosis, cirrhosis, multiple sclerosis, glomerulonephritis, graft versus host disease, diabetes, inflammatory bowel disease, shock, invertebral disc degeneration, stroke, osteopenia and periodontal diseases.

61. The method of claim 60, wherein the metalloproteinase-related disorder is osteoarthritis.

62. The method of any of claims 57-61, wherein the mammal is a human.

63. A pharmaceutical composition made from a polymorph as claimed in any one of claims 1-42 or 48-50 and a pharmaceutically acceptable carrier.

64. Use of a polymorph as claimed in any one of claims 1-42 or 48-50 for preparing a medicament for treating a metalloproteinase-related disorder.
